(19) United States
(12) Patent Application Publication
(10) Pub. No.: US 2023/0343415 A1
(43) Pub. Date: Oct. 26, 2023

(54) GENERATING CLUSTER-SPECIFIC-SIGNAL CORRECTIONS FOR DETERMINING NUCLEOTIDE-BASE CALLS

(71) Applicant: Illumina Software, Inc., San Diego, CA (US)

(72) Inventors: Eric Jon Ojard, San Francisco, CA (US); John S. Vieceli, Encinitas, CA (US); Gavin Derek Parnaby, Laguna Niguel, CA (US); Bo Lu, San Diego, CA (US); Rami Mehio, San Diego, CA (US)

(21) Appl. No.: 18/059,326

(22) Filed: Nov. 28, 2022

Related U.S. Application Data
(60) Provisional application No. 63/285,187, filed on Dec. 2, 2021.

(51) Int. Cl. G16B 30/10; C12Q 1/6869

(52) U.S. Cl. CPC G16B 30/10; C12Q 1/6869

(57) ABSTRACT

This disclosures describes embodiments of methods, systems, and non-transitory computer readable media that accurately and efficiently estimate the effects of phasing and pre-phasing for a particular cluster of oligonucleotides and determining a cluster-specific-phasing correction for the cluster. For instance, the disclosed systems can dynamically identify clusters of oligonucleotides exhibiting error-inducing sequences that frequently cause phasing or pre-phasing. When the disclosed systems detect signals during cycles at read positions following such an error-inducing sequence, the disclosed systems can generate cluster-specific-phasing coefficients and correct the signals according to such cluster-specific-phasing coefficients. For instance, the disclosed system can utilize a linear equalizer, decision feedback equalizer, or a maximum likelihood sequence estimator to generate cluster-specific-phasing coefficients.

### CROSS-REFERENCE TO RELATED APPLICATIONS

The present application claims the benefit of, and priority to, U.S. Provisional Application No. 63/285,187, entitled “GENERATING CLUSTER-SPECIFIC-SIGNAL CORRECTIONS FOR DETERMINING NUCLEOTIDE-BASE CALLS,” filed on Dec. 2, 2021. The aforementioned application is hereby incorporated by reference in its entirety.

### BACKGROUND

In recent years, biotechnology firms and research institutions have improved hardware and software platforms used for determining a sequence of nucleotide bases in a sample genome or other nucleic-acid polymer. For instance, some existing nucleic-acid-sequencing platforms determine individual nucleotide bases of nucleic-acid sequences by using conventional Sanger sequencing or sequencing-by-synthesis (SBS). When using SBS, existing platforms can monitor thousands, tens of thousands, or more oligonucleotides that are grouped into clusters and synthesized in parallel to detect more accurate nucleotide-base calls. For instance, a camera in SBS platforms can capture images of irradiated fluorescent tags from nucleotide bases incorporated into such clustered and synthesized oligonucleotides. After capturing the images, existing SBS platforms send image data to a computing device with sequencing-data-analysis software to determine a nucleotide-base sequence for a genome or other nucleic-acid polymer. For instance, the sequencing-data-analysis software can determine the nucleotide bases with tags that irradiate in a given image based on the light signal captured in the image data. By cyclically incorporating nucleotide bases into the oligonucleotides and capturing images of the emitted light signals in various sequencing cycles, the SBS platforms can determine nucleotide reads corresponding to particular clusters and determine the sequence of nucleotide bases present in a whole genome sample or other samples of nucleic-acid polymers.

Despite these recent advances, existing nucleic-acid-sequencing platforms and sequencing-data-analysis software (together and hereinafter, “existing sequencing systems”) often suffer from technical limitations that impede the accuracy, applicability, and efficiency of detecting and correcting signals for phasing. When an existing nucleic-acid-sequencing platform executes a cycle to incorporate and detect a nucleotide base for oligonucleotides of various clusters, the platform often incorporates and detects some nucleotide bases out of phase. When phasing and pre-phasing occur, a nucleic-acid-sequencing platform respectively incorporates a nucleotide base corresponding to a previous cycle (phasing) or a nucleotide base corresponding to a subsequent cycle (pre-phasing). Because of phasing or pre-phasing, the nucleic-acid-sequencing platform captures images of light signals from clusters with a mix of incorporated nucleotide bases for a current cycle—as well as incorporated nucleotide bases corresponding to previous or subsequent cycles. Existing sequencing systems frequently fail to accurately detect and correct for such phasing and pre-phasing effects and, consequently, sometimes determine an incorrect nucleotide-base call for a nucleotide read corresponding to a cluster at a particular cycle. Even when existing sequencing systems generate correct nucleotide-base calls, such systems can generate base calls for reads with lower quality sequencing metrics due in part to phasing and pre-phasing. For instance, existing sequencing systems that capture mixed signals at read positions following certain repetitive nucleotide sequences often generate base calls with lower quality scores, such as Phred quality scores (e.g., below Q30).

Existing sequencing systems frequently attempt to circumvent the inaccuracies caused by phasing and pre-phasing mentioned above. But these systems are often rigid and rely on a one-size-fits-all approach. For example, conventional sequencing systems often rely on global phasing and global pre-phasing corrections to maximize the chastity of intensity data for each cycle. Chastity values indicate a ratio of the brightest base intensity divided by the sum of the brightest and the second brightest base intensities. The use of global phasing and global pre-phasing corrections limits the effectiveness of phasing correction to signals to large sections of a slide (e.g., a flow cell). Indeed, conventional sequencing systems often fail to account for variability at the cluster level. For instance, a first cluster within a section (e.g., tile) of a slide may exhibit significant phasing effects, a second cluster within the section may exhibit significant pre-phasing effects, and a third cluster within the same section may exhibit little-to-no phasing or pre-phasing. Thus, conventional sequencing systems that rely on global phasing and global pre-phasing corrections often fail to account for nuanced variation within clusters.

Furthermore, conventional sequencing systems often include limited storage resources and other computational resources to efficiently capture and analyze image data of various clusters. In particular, as part of applying phasing corrections, conventional sequencing systems frequently store and analyze sequencing image data or sequencing intensity data. To illustrate, conventional sequencing systems often collect signal data for each cycle, store the data, and analyze the data. Due to the storage load required save such image data cycle after cycle, it is often impractical to store and process image or signal data utilizing the memory devices of sequencing machines. To illustrate, conventional systems often collect signal data for each cycle, store the data on a sequencing device, transfer the data to a server, store the data in the server, and process the data from each cycle on the server. Thus, not only do conventional systems inefficiently utilize resources, but they also introduce significant latencies by transferring and processing the signaling data.

These, along with additional problems and issues exist in existing sequencing systems.

### BRIEF SUMMARY

This disclosure describes one or more embodiments of systems, methods, and non-transitory computer readable storage media that solve one or more of the problems described above or provide other advantages over the art. In particular, the disclosed system can accurately and efficiently estimate the effects of phasing and pre-phasing for a particular cluster of oligonucleotides and determine a cluster-specific-phasing correction for the cluster. For instance, the disclosed systems can dynamically identify clusters of oligonucleotides exhibiting error-inducing sequences that frequently cause phasing or pre-phasing. When the disclosed systems detect signals during cycles at read positions following such an error-inducing sequence, the disclosed systems can generate cluster-specific-phasing coefficients and correct the signals according to such cluster-specific-phasing coefficients. For instance, the disclosed system can utilize a linear equalizer, decision feedback equalizer, a maximum likelihood sequence estimator, or a machine learning model to generate cluster-specific-phasing coefficients. In some cases, the disclosed system can accordingly identify read positions following error-inducing sequences and generate cluster-specific-phasing coefficients with little-to-no buffering in near-real time on sequencing devices.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description will describe various embodiments with additional specificity and detail through the use of the accompanying drawings, which are summarized below.

FIG. 1 illustrates an environment in which a cluster-aware-base-calling system can operate in accordance with one or more embodiments of the present disclosure.

FIG. 2A illustrates an example read pileup indicating incorrect base-calls resulting from phasing and pre-phasing before cluster-specific-phasing correction in accordance with one or more embodiments of the present disclosure.

FIG. 2B illustrates a schematic diagram demonstrating phasing and pre-phasing in accordance with one or more embodiments of the present disclosure.

FIG. 3 illustrates an overview diagram of the cluster-aware-base-calling system determining a cluster-specific-phasing correction and determining a nucleotide-base call based on adjusting a signal based on the cluster-specific-phasing correction in accordance with one or more embodiments of the present disclosure.

FIG. 4 illustrates cluster-aware-base-calling system identifying an error-inducing sequence based on analyzing signals from previous cycles in accordance with one or more embodiments of the present disclosure.

FIG. 5 illustrates the cluster-aware-base-calling system determining a cluster-specific-phasing coefficient and a cluster-specific-pre-phasing coefficient in accordance with one or more embodiments of the present disclosure.

FIG. 6 illustrates an example phasing model the cluster-aware-base-calling system utilizes to estimate cluster-specific-phasing corrections in accordance with one or more embodiments of the present disclosure.

FIGS. 7A-7C illustrate the cluster-aware-base-calling system utilizing various receiver types including a linear equalizer, a decision feedback equalizer, and a maximum likelihood sequence estimation equalizer to determine cluster-specific-phasing corrections in accordance with one or more embodiments of the present disclosure.

FIGS. 8A-8B illustrate graphs indicating metrics showing the cluster-aware-base-calling system improves base-call accuracy and various secondary sequencing metrics by adjusting signals based on cluster-specific-phasing corrections in accordance with one or more embodiments of the present disclosure.

FIG. 9 illustrates a series of acts for determining a cluster-specific-phasing correction and determining a nucleotide-base call based on adjusting a signal based on the cluster-specific-phasing correction in accordance with one or more embodiments of the present disclosure.

FIG. 10 illustrates a block diagram of an example computing device in accordance with one or more embodiments of the present disclosure.

### DETAILED DESCRIPTION

This disclosure describes one or more embodiments of a cluster-aware-base-calling system that estimates phasing errors on a per-cluster basis. In particular, the cluster-aware-base-calling system identifies sequences that frequently induce signal deterioration. For example, the cluster-aware-base-calling system can identify homopolymer sequences, G-quadruplex sequences, or other error-inducing sequences within a nucleotide-fragment read corresponding to a cluster of oligonucleotides. The cluster-aware-base-calling system can further determine coefficients that estimate effects of phasing and pre-phasing on signals for nucleotide bases from a current cycle. The cluster-aware-base-calling system utilizes the cluster-specific-phasing coefficients to correct signal intensities from which nucleotide-base calls are made. By correcting for estimated phasing or pre-phasing on a per-cluster basis, the cluster-aware-base-calling system can analyze the corrected signal intensities to generate more accurate nucleotide-base-calls.

To illustrate, in one or more embodiments, the cluster-aware-base-calling system identifies, for a cluster of oligonucleotides, a read position following an error-inducing sequence within one or more nucleotide-fragment reads. The cluster-aware-base-calling system can further detect a signal from labeled nucleotide bases within the cluster of oligonucleotides during a cycle corresponding to the read position. For the same cluster, the cluster-aware-base-calling system determines a cluster-specific-phasing correction to correct the signal for estimated phasing and estimated pre-phasing. The cluster-aware-base-calling system may then adjust the signal based on the cluster-specific-phasing correction. Based on the adjusted signal, the cluster-aware-base-calling system can determine a nucleotide-base call for the read position corresponding to the cluster of oligonucleotides.

As mentioned, in some cases, the cluster-aware-base-calling system identifies a read position following an error-inducing sequence within one or more nucleotide-fragment reads corresponding to a cluster of oligonucleotides. Such error-inducing sequences can trigger systematic sequencing errors that negatively impact the quality and accuracy of sequencing runs. To reduce the number of clusters for which a cluster-specific-phasing correction is determined, in some embodiments, the cluster-aware-base-calling system limits the computing resources used for phasing correction by determining such cluster-specific-phasing corrections only for read positions of a cluster following error-inducing sequences. Examples error-inducing sequences can include one or more repeated nucleotide bases, such as homopolymers, or sequence motifs, such as guanine quadruplexes. The cluster-aware-base-calling system can analyze signals from a cluster of oligonucleotides from previous sequencing cycles to determine the presence of an error-inducing sequence within a nucleotide-fragment read corresponding to the cluster.

After or while identifying an error-inducing sequence corresponding to a cluster of oligonucleotides, the cluster-aware-base-calling system can detect a signal from labeled nucleotide bases within the cluster of oligonucleotides during a cycle corresponding to the read position. As mentioned, SBS sequencing systems capture images of irradiated fluorescent tags from labeled nucleotide bases as labeled nucleotide bases are iteratively incorporated into a cluster's oligonucleotides. The cluster-aware-base-calling system can detect signals from the labeled nucleotide bases specifically for a cycle corresponding to one or more read positions—following the error-inducing sequence—and identify such signals as targets for cluster-specific-phasing correction.

After identifying a signal corresponding to a relevant read position following an error-inducing sequence, the cluster-aware-base-calling system can determine a cluster-specific-phasing correction to correct the signal for estimated phasing and estimated pre-phasing. As mentioned, systematic sequencing errors can include phasing and pre-phasing in which nucleotide bases are incorporated late or early, respectively. In some embodiments, the cluster-aware-base-calling system determines the cluster-specific-phasing correction by determining (i) one or more cluster-specific-phasing coefficients corresponding to nucleotide bases for one or more previous cycles and (ii) one or more cluster-specific pre-phasing coefficients corresponding to nucleotide bases for one or more subsequent cycles. The cluster-aware-base-calling system can further determine the cluster-specific-phasing correction based on the cluster-specific-phasing coefficient and the cluster-specific-pre-phasing coefficient.

To determine such cluster-specific phasing and pre-phasing coefficients, the cluster-aware-base-calling system can utilize a number of models or algorithms. For example, in some cases, the cluster-aware-base-calling system utilizes a real-time linear equalizer to estimate the cluster-specific-phasing coefficient and the cluster-specific pre-phasing coefficient. The linear equalizer is computationally efficient and requires little-to-no buffering compared to alternative coefficient algorithms. Accordingly, the cluster-aware-base-calling system can implement the linear equalizer on a sequencing device to estimate cluster-specific-phasing corrections in real time. Alternatively, in some embodiments, the cluster-aware-base-calling system utilizes a decision feedback equalizer, maximum likelihood equalizer, or a machine learning model instead of, or in addition to, the linear equalizer to estimate cluster-specific-phasing corrections.

After determining a cluster-specific-phasing correction, the cluster-aware-base-calling system can adjust the signal based on the cluster-specific-phasing correction. In particular, the cluster-aware-base-calling system estimates a cluster-specific-phasing correction for a cluster having an error-inducing sequence and applies the cluster-specific-phasing correction to the signal from the cluster. In some embodiments, the cluster-aware-base-calling system also determines, for a set of clusters, a multi-cluster-phasing correction to correct for sequencing errors across the set of clusters. Such a multi-cluster-phasing correction may include, for instance, a global phasing coefficient and a global pre-phasing coefficient as part of a global phasing correction for clusters in a tile of a flow cell. The cluster-aware-base-calling system can also adjust the signal for a cluster based on a combination of the cluster-specific-phasing correction and the multi-cluster-phasing correction.

The cluster-aware-base-calling system provides several technical benefits relative to existing sequencing systems. In particular, the cluster-aware-base-calling system can improve the accuracy, tailored applicability, and efficiency of phasing corrections relative to existing sequencing systems. As mentioned, the cluster-aware-base-calling system determines both phasing corrections for signals and nucleotide-base calls based on such corrected signals—with better accuracy than existing sequencing systems. By determining and applying a cluster-specific-phasing correction to a signal for certain read positions corresponding to a cluster, the cluster-aware-base-calling system can reduce the negative impact of homopolymer sequences, G-quadruplex sequences, or other error-inducing sequences on the accuracy of predicted nucleotide-base calls. Furthermore, by adjusting a signal for estimated phasing and pre-phasing on a per-cluster basis, the cluster-aware-base-calling system can reduce the amount of noise caused by phasing or pre-phasing effects in the signal from the incorporated nucleotide bases of a specific cluster of oligonucleotides. Simply put, the cluster-aware-base-calling system can identify and correct for phasing and pre-phasing effects for a particular cluster better than existing sequencing systems.

As further shown below, by correcting signals used to generate nucleotide-base calls, the cluster-aware-base-calling system also improves secondary sequencing metrics, such as better quality metrics for base-call data, and improves the baseline for estimating or calibrating metrics for a sequencing device, such as by improving signal to noise ratio (SNR) metrics. Because cluster-specific-phasing correction improves signals used to generate nucleotide-base calls, the cluster-aware-base-calling system can also reduce the impact of correlated error-inducing sequences (e.g., sequences that trigger systematic sequencing errors) that compound one after another to negatively affect the performance of downstream nucleotide-base calling tools, such as mapper-and-alignment components of a call-generation model (e.g., DRAGEN) or variant-caller components of the call-generation model.

In addition to being more accurate, the cluster-aware-base-calling system creates a phasing correction that is more tailored to cluster-specific sequencing errors than existing sequencing systems. In contrast to existing systems that apply phasing corrections across groups of clusters or all clusters of oligonucleotides, the cluster-aware-base-calling system determines cluster-specific-phasing coefficients. Indeed, in some cases, the cluster-aware-base-calling system selectively determines and applies cluster-specific-phasing corrections to signals at post-error-inducing-sequence read positions for certain clusters and applies multi-cluster-phasing corrections (without cluster-specific-phasing corrections) to signals at read position for certain other clusters that lack such error-inducing sequences. Thus, even as clusters can become more problematic as sequencing progresses—as phasing and pre-phasing effects tend to increase during a sequencing run—the cluster-aware-base-calling system adjusts the cluster-specific-phasing corrections to make corresponding adjustments to nucleotide-base calls.

As indicated above, in some embodiments, the cluster-aware-base-calling system can improve the computing efficiency of correcting signals for phasing and pre-phasing effects relative to alternative computational models for phasing correction. In contrast to a computational model that would process and correct for phasing and pre-phasing for each cluster across every cycle, the cluster-aware-base-calling system reduces the amount of computing resources utilized by processing and correcting signals from labeled nucleotide bases following error-inducing sequences. As noted above, in some embodiments, the cluster-aware-base-calling system limits the computing resources used for phasing correction by determining cluster-specific-phasing corrections only for read positions of a cluster following error-inducing sequences.

Furthermore, by utilizing a linear-equalizer based approach to determine phasing corrections, in some cases, the cluster-aware-base-calling system can estimate the cluster-specific-phasing corrections in real (or near-real) time on a sequencing device. Some existing sequencing systems consume significantly more computing memory on a sequencing machine (or other computing device) by saving image data for the signals of all clusters for an entire sequencing run and determining phasing corrections only after the sequencing run has finished. In contrast, in certain embodiments, the cluster-aware-base-calling system discards data for a signal after applying a cluster-specific-phasing correction and/or a multi-cluster-phasing correction. In at least one embodiment, by processing and correcting signals for phasing and pre-phasing effects on the sequencing device, the cluster-aware-base-calling system can reduce the amount of storage, communication, and computing resources typically required to communicate data to a central location, process the data, and communicate the results.

As illustrated by the foregoing discussion, the present disclosure utilizes a variety of terms to describe features and advantages of the cluster-aware-base-calling system. Additional detail is now provided regarding the meaning of such terms. For example, as used herein, the term “cluster” refers to a group of oligonucleotides or nucleic-acid segments from a sample genome organized on a nucleotide-sample slide. In particular, a cluster includes tens, hundreds, thousands, or more copies of a cloned or the same DNA or RNA segment. For example, in one or more embodiments, a cluster includes a grouping of oligonucleotides immobilized in a section of a nucleotide-sample slide (e.g., a flow cell). In some embodiments, clusters are evenly spaced or organized in a systematic structure within a patterned nucleotide-sample slide. By contrast, in some cases, clusters are randomly organized within a non-patterned nucleotide-sample slide.

As used herein, the term “oligonucleotide” refers to an oligomer or other polymer of nucleotides or mimetics. In particular, an oligonucleotide can include a synthetic or natural molecule comprising a sequence of covalently linked nucleotides formed by a modified phosphodiester or phosphodiester bond between the 3′ position of the pentose in a nucleotide and the 5′ position of the pentose in a nucleotide adjacent. For example, an oligonucleotide can include a short DNA or RNA molecule annealed to a single-stranded polynucleotide to be analyzed or sequenced as part of SBS sequencing.

As further used herein, the term “nucleotide-sample slide” refers to a plate or slide comprising oligonucleotides for sequencing nucleotide segments for sample genomes or other sample nucleic-acid polymers. In particular, a nucleotide-sample slide can refer to a slide containing fluidic channels through which reagents and buffers can travel as part of sequencing. For example, in one or more embodiments, a nucleotide-sample slide includes a flow cell (e.g., a patterned flow cell or non-patterned flow cell) comprising small fluidic channels and short oligonucleotides complementary to adaptor sequences. As indicated above, a nucleotide-sample slide can include wells (e.g., nanowells) comprising clusters of oligonucleotides.

As used herein, a flow cell or other nucleotide-sample slide can (i) include a device having a lid extending over a reaction structure to form a flow channel therebetween that is in communication with a plurality of reaction sites of the reaction structure and (ii) include a detection device that is configured to detect designated reactions that occur at or proximate to the reaction sites. A flow cell or other nucleotide-sample slide may include a solid-state light detection or “imaging” device, such as a Charge-Coupled Device (CCD) or Complementary Metal-Oxide Semiconductor (CMOS) (light) detection device. As one specific example, a flow cell may be configured to fluidically and electrically couple to a cartridge (having an integrated pump), which may be configured to fluidically and/or electrically couple to a bioassay system. A cartridge and/or bioassay system may deliver a reaction solution to reaction sites of a flow cell according to a predetermined protocol (e.g., sequencing-by-synthesis), and perform a plurality of imaging events. For example, a cartridge and/or bioassay system may direct one or more reaction solutions through the flow channel of the flow cell, and thereby along the reaction sites. At least one of the reaction solutions may include four types of nucleotides having the same or different fluorescent labels. The nucleotides may bind to the reaction sites of the flow cell, such as to corresponding oligonucleotides at the reaction sites. The cartridge and/or bioassay system may then illuminate the reaction sites using an excitation light source (e.g., solid-state light sources, such as light-emitting diodes (LEDS)). The excitation light may provide emission signals (e.g., light of a wavelength or wavelengths that differ from the excitation light and, potentially, each other) that may be detected by the light sensors of the flow cell.

As used herein, the term “read position” refers to a location or coordinate on nucleotide-fragment read. In particular, a read position includes a location along a nucleotide-fragment read to which a labeled nucleotide has been added. For example, a read position can indicate a position within a nucleotide-fragment read at which a most-recently added labeled nucleotide to corresponding oligonucleotides within a cluster when a camera captures an image of a nucleotide-sample slide or a section of the nucleotide-sample slide.

As used herein, the term “nucleotide-fragment read” refers to an inferred sequence of one or more nucleotide bases (or nucleobase pairs) from all or part of a sample nucleotide sequence. In particular, a nucleotide-fragment read includes a determined or predicted sequence of nucleotide-base calls for a nucleotide fragment (or group of monoclonal nucleotide fragments) from a sequencing library corresponding to a genome sample. For example, in some cases, a sequencing device determines a nucleotide-fragment read by generating nucleotide-base calls for nucleotide bases passed through a nanopore of a nucleotide-sample slide, determined via fluorescent tagging, or determined from a cluster in a flow cell.

As used herein, the term “error-inducing sequence” refers to a nucleotide-base sequence or corresponding chemical structure that induces or triggers a sequencing error. In particular, an error-inducing sequence refers to a nucleotide-base sequence that triggers systematic sequencing errors (SSE) during SBS sequencing. For instance, an error-inducing sequence can cause dephasing by inducing a sequencing device to add or incorporate an incorrect labeled nucleotide bases at the wrong cycle. For example, error-inducing sequences can include homopolymers of a same nucleotide base, a guanine quadruplex, a variable number tandem repeat (VNTR), a dinucleotide-repeat sequence, a tri-nucleotide-repeat sequence, an inverted-repeat sequence, a minisatellite sequence, a microsatellite sequence, a palindromic sequence, or other sequence.

As used herein, the term “signal” refers to refers to a signal emitted, reflected, or otherwise communicated from a labeled nucleotide base or a group of labeled nucleotide bases (e.g., labeled nucleotide bases added to a cluster of oligonucleotides). In particular, a signal can refer to a signal indicating the type of nucleotide base. For example, a signal can include a light signal emitted or reflected from a fluorescent tag of a nucleotide base or fluorescent tags of multiple nucleotide bases incorporated into oligonucleotides. In some implementations, the cluster-aware-base-calling system triggers the signal through an external stimulus, such as a laser or other light source. In some cases, the cluster-aware-base-calling system triggers the signal through some internal stimuli. Further, in some embodiments, the cluster-aware-base-calling system observes the signal using a filter applied when capturing an image of the nucleotide-sample slide (e.g., section of the nucleotide-sample slide). As suggested above, in certain instances, a signal includes an aggregate of the signals provided by each labeled nucleotide base added to individual oligonucleotides in a cluster of oligonucleotides.

As used herein, the term “labeled nucleotide base” refers to a nucleotide base having a fluorescent or light-based indicator of the classification of the nucleotide base. In particular, a labeled nucleotide base can refer to a nucleotide base that incorporates a fluorescent or light-based indicator to identify the type of nucleotide base (e.g., adenine, cytosine, thymine, or guanine). For example, in one or more embodiments, a labeled nucleotide base includes a nucleotide base having a fluorescent tag that emits a signal that identifies the nucleotide-base type.

As used herein, the term “sequencing cycle” (or “cycle”) refers to an iteration of adding or incorporating a nucleotide base to an oligonucleotide or an iteration of adding or incorporating nucleotide bases to oligonucleotides in parallel. In particular, a cycle can include an iteration of taking an analyzing one or more images with data indicating individual nucleotide bases added or incorporated into an oligonucleotide or to oligonucleotides in parallel. Accordingly, cycles can be repeated as part of sequencing a nucleic-acid polymer (e.g., sample genome). For example, in one or more embodiments, each sequencing cycle involves either single nucleotide-fragment reads in which DNA or RNA strands are read in only a single direction or paired-end reads in which DNA or RNA strands are read from both ends. Further, in certain cases, each sequencing cycle involves a camera taking an image of the nucleotide-sample slide or multiple sections of the nucleotide-sample slide to generate image data for determining a particular nucleotide base added or incorporated into particular oligonucleotides. Following the image capture stage, a sequencing system can remove certain fluorescent labels from incorporated nucleotide bases and perform another sequencing cycle until the nucleic-acid polymer has been completely sequenced. In one or more embodiments, a sequencing cycle includes a cycle within a Sequencing By Synthesis (SBS) run.

As used herein, the term “cluster-specific-phasing correction” refers to a process or function that, when applied, adjusts a signal from labeled nucleotides bases within a particular cluster of oligonucleotides to correct for estimated phasing or pre-phasing. In particular, a cluster-specific-phasing correction can include an algorithm or function by which a signal from a cluster should be adjusted to correct for the estimated effects of estimated phasing or pre-phasing using a Fourier transform.

As used herein, the term “phasing” refers to an instance of (or rate at which) labeled nucleotide bases are incorporated behind a particular sequencing cycle. Phasing includes an instance of (or rate at which) labeled nucleotide bases within a cluster are asynchronously incorporated behind other labeled nucleotide bases within a cluster for a particular sequencing cycle. In particular, during SBS, each DNA strand in a cluster extends incorporation by one nucleotide base per cycle. One or more oligonucleotide strands within the cluster may become out of phase with the current cycle. Phasing occurs when nucleotide bases for one or more oligonucleotides within a cluster fall behind one or more cycles of incorporation. For example, a nucleotide sequence from a first location to a third location may be CTA. In this example, the C nucleotide should be incorporated in a first cycle, T in the second cycle, and A in the third cycle. When phasing occurs during the second sequencing cycle, one or more labeled C nucleotides are incorporated instead of a T nucleotide. Relatedly, as used herein, the term “pre-phasing” refers to an instance of (or rate at which) one or more nucleotide bases are incorporated ahead of a particular cycle. Pre-phasing includes an instance of (or rate at which) labeled nucleotide bases within a cluster are asynchronously incorporated ahead other labeled nucleotide bases within a cluster for a particular sequencing cycle. To illustrate, when pre-phasing occurs during the second sequencing cycle in the example above, one or more labeled A nucleotides are incorporated instead of a T nucleotide.

As used herein, the term “cluster-specific-phasing coefficient” refers to a factor or value that estimates or measures cluster-specific phasing on a signal for a cluster. In particular, a cluster-specific-phasing coefficient estimates the effects of phasing for a cluster within a given sequencing cycle. For example, a cluster-specific-phasing coefficient can indicate the effect a nucleotide base for a previous cycle has on a signal from labeled nucleotide bases for a current cycle. To illustrate, in the example described above, a cluster-specific-phasing coefficient can estimate the effect of phasing from the C nucleotide that is incorporated instead of a T nucleotide during the second sequencing cycle.

Relatedly, the term “cluster-specific-pre-phasing coefficient” refers to a factor or value that estimates or measures cluster-specific pre-phasing on a signal for a cluster. In particular, a cluster-specific-pre-phasing coefficient estimates the effects of pre-phasing for a cluster within a given sequencing cycle. For example, a cluster-specific-pre-phasing coefficient can indicate the effect a nucleotide base for a subsequent cycle has on a signal from labeled nucleotide bases for a current cycle. To illustrate, in the example described above, a cluster-specific-pre-phasing coefficient estimates the effect of pre-phasing from the A nucleotide that is incorporated instead of a T nucleotide during the second sequencing cycle.

As used herein, the term “nucleotide-base call” (or simply “base call”) refers to a determination or prediction of a particular nucleotide base (or nucleotide-base pair) for a genomic coordinate of a sample genome or for an oligonucleotide during a sequencing cycle. In particular, a nucleotide-base call can indicate (i) a determination or prediction of the type of nucleotide base that has been incorporated within an oligonucleotide on a nucleotide-sample slide (e.g., read-based nucleotide-base calls) or (ii) a determination or prediction of the type of nucleotide base that is present at a genomic coordinate or region within a genome, including a variant call or a non-variant call in a digital output file. In some cases, for a nucleotide-fragment read, a nucleotide-base call includes a determination or a prediction of a nucleotide base based on intensity values resulting from fluorescent-tagged nucleotides added to an oligonucleotide of a nucleotide-sample slide (e.g., in a cluster of a flow cell). Alternatively, a nucleotide-base call includes a determination or a prediction of a nucleotide base from chromatogram peaks or electrical current changes resulting from nucleotides passing through a nanopore of a nucleotide-sample slide. By contrast, a nucleotide-base call can also include a final prediction of a nucleotide base at a genomic coordinate of a sample genome for a variant call file or other base-call-output file-based on nucleotide-fragment reads corresponding to the genomic coordinate. Accordingly, a nucleotide-base call can include a base call corresponding to a genomic coordinate and a reference genome, such as an indication of a variant or a non-variant at a particular location corresponding to the reference genome. Indeed, a nucleotide-base call can refer to a variant call, including but not limited to, a single nucleotide variant (SNV), an insertion or a deletion (indel), or base call that is part of a structural variant. As suggested above, a single nucleotide-base call can be an adenine (A) call, a cytosine (C) call, a guanine (G) call, or a thymine (T) call.

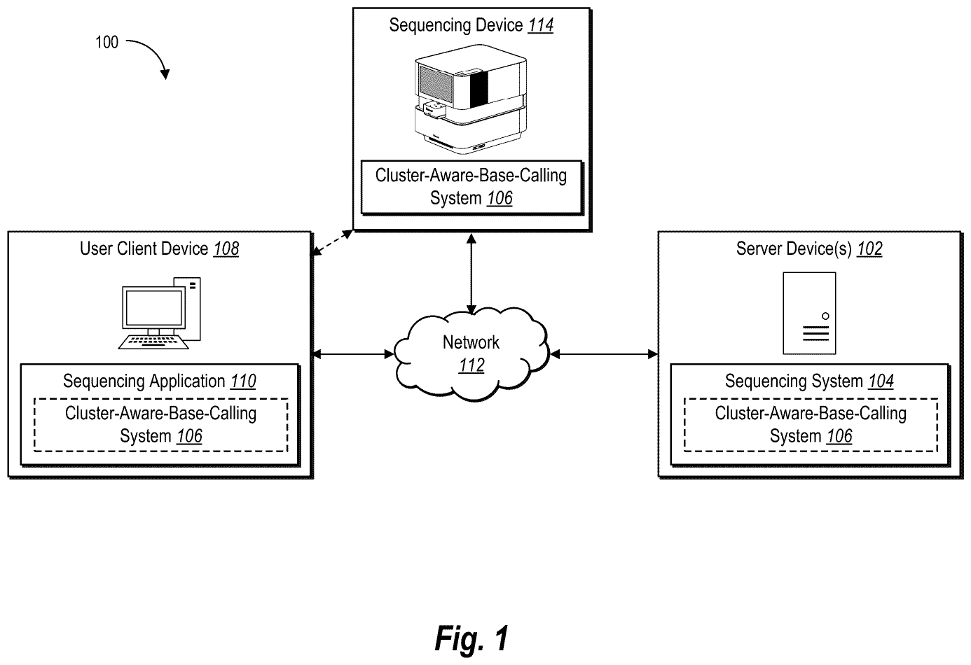

Additional detail will now be provided regarding a cluster-aware-base-calling system in relation to illustrative figures portraying example embodiments and implementations of the cluster-aware-base-calling system. For example, FIG. 1 illustrates a schematic diagram of a system environment (or “environment”) 100 in which a cluster-aware-base-calling system 106 operates in accordance with one or more embodiments. As illustrated, the environment 100 includes one or more server device(s) 102 connected to a user client device 108 and a sequencing device 114 via a network 112. While FIG. 1 shows an embodiment of the cluster-aware-base-calling system 106, alternative embodiments and configurations are possible.

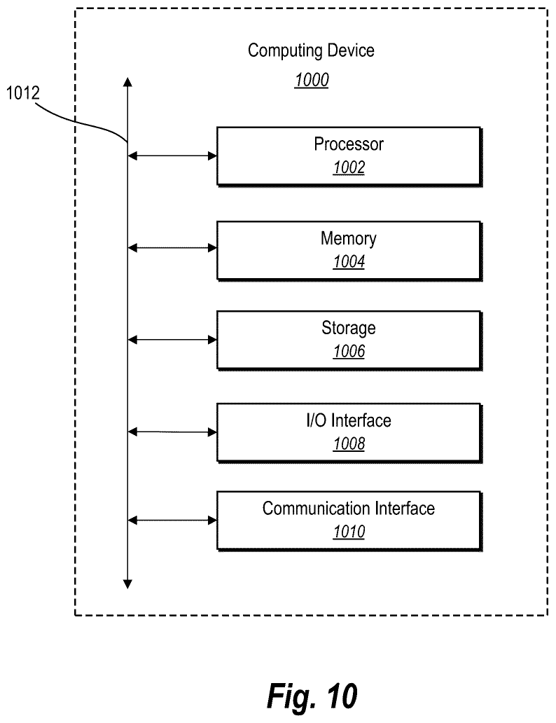

As further shown in FIG. 1, the server device(s) 102, the user client device 108, and the sequencing device 114 are connected via the network 112. Each of the components of the environment 100 can communicate via the network 112. The network 112 comprises any suitable network over which computing devices can communicate. Example networks are discussed in additional detail below in relation to FIG. 10.

As shown in FIG. 1, the environment 100 includes the sequencing device 114. The sequencing device 114 comprises a device for sequencing a whole genome or other nucleic-acid polymer. In some embodiments, the sequencing device 114 analyzes samples to generate data utilizing computer implemented methods and systems described herein either directly or indirectly on the sequencing device 114. In one or more embodiments, the sequencing device 114 utilizes Sequencing By Synthesis (SBS) to sequence whole genomes or other nucleic-acid polymers. As shown, in some embodiments, the sequencing device 114 bypasses the network 112 and communicates directly with the user client device 108.

As further depicted by FIG. 1, the environment 100 includes the server device(s) 102. The server device(s) 102 may generate, receive, analyze, store, receive, and transmit electronic data, such as data for sequencing nucleic-acid polymers. The server device(s) 102 may receive data from the sequencing device 114. For example, the server device(s) 102 may gather and/or receive sequencing data including nucleotide-base call data, quality data, and other data relevant to sequencing nucleic-acid polymers. The server device(s) 102 may also communicate with the user client device 108. In particular, the server device(s) 102 can send nucleic-acid polymer sequences, error data, and other information to the user client device 108. In some embodiments, the server device(s) 102 comprise distributed servers, where the server device(s) 102 include a number of server devices distributed across the network 112 and located in different physical locations. The server device(s) 102 can comprise a content server, an application server, a communication server, a web-hosting server, or another type of server.

As further shown in FIG. 1, the server device(s) 102 can include the sequencing system 104. Generally, the sequencing system 104 analyzes sequencing data received from the sequencing device 114 to determine nucleotide sequences for whole genomes or other nucleic-acid polymers. For example, the sequencing system 104 can receive raw data (e.g., base-call data for nucleotide-fragment reads) from the sequencing device 114 and determine a nucleic acid sequence for a sample genome. To illustrate, the sequencing system 104 can receive nucleotide-fragment reads from the sequencing device 114, and the sequencing system 104 generates nucleotide-base calls for a sample genome from the nucleotide-fragment reads. In some embodiments, the sequencing system 104 determines the sequences of nucleotide bases in DNA and/or RNA. In addition to processing and determining sequences for nucleic-acid polymers, the sequencing system 104 also analyzes sequencing data to detect irregularities in individual or multiple sequencing cycles.

As illustrated in FIG. 1, the sequencing device 114 includes the cluster-aware-base-calling system 106. Generally, the cluster-aware-base-calling system 106 estimates a cluster-specific-phasing correction to correct a signal for estimated phasing and pre-phasing. More specifically, in some embodiments, the cluster-aware-base-calling system 106 identifies a read position following an error-inducing sequence within one or more nucleotide-fragment reads. The cluster-aware-base-calling system 106 further detects a signal from labeled nucleotide bases within the cluster of oligonucleotides during a cycle corresponding to the read position. The cluster-aware-base-calling system 106 determines a cluster-specific-phasing correction to correct the signal for estimated phasing and estimated pre-phasing. The cluster-aware-base-calling system 106 adjusts the signal based on the cluster-specific-phasing correction and determines a nucleotide-base-call for the read position corresponding to the cluster of oligonucleotides based on the adjusted signal.

The environment 100 illustrated in FIG. 1 further includes the user client device 108. The user client device 108 can generate, store, receive, and send digital data. In particular, the user client device 108 can receive sequencing data from the sequencing device 114. Furthermore, the user client device 108 may communicate with the server device(s) 102 to receive nucleotide-base calls, nucleotide sequences, and reports of irregularities within a sequencing run. The user client device 108 can present sequencing data to a user associated with the user client device 108.

The user client device 108 illustrated in FIG. 1 may comprise various types of client devices. For example, in some embodiments, the user client device 108 includes non-mobile devices, such as desktop computers or servers, or other types of client devices. In yet other embodiments, the user client device 108 includes mobile devices, such as laptops, tablets, mobile telephones, smartphones, etc. Additional details with regard to the user client device 108 are discussed below with respect to FIG. 10.

As further illustrated in FIG. 1, the user client device 108 includes a sequencing application 110. The sequencing application 110 may be a web application or a native application on the user client device 108 (e.g., a mobile application, desktop application, etc.). The sequencing application 110 can comprise instructions that (when executed) cause the user client device 108 to receive or request data from the cluster-aware-base-calling system 106 and present sequencing data. Furthermore, the sequencing application 110 can comprise instructions that (when executed) cause the user client device 108 to provide a graphical visualization of a read pileup or read alignment for a sample genome.

As further illustrated in FIG. 1, the cluster-aware-base-calling system 106 may be located on the user client device 108 as part of the sequencing application 110. As illustrated, in some embodiments, the cluster-aware-base-calling system 106 is implemented by (e.g., located entirely or in part) on the user client device 108. In yet other embodiments, the cluster-aware-base-calling system 106 is implemented by one or more other components of the environment 100. In particular, the cluster-aware-base-calling system 106 can be implemented in a variety of different ways across the server device(s) 102, the user client device 108, and the sequencing device 114. In one example, the cluster-aware-base-calling system 106 is located in part on the sequencing device 114 and also the server device(s) 102. In particular, the cluster-aware-base-calling system 106 can adjust the signal based on the cluster-specific-phasing correction on the sequencing device 114 and determine the nucleotide-base call for the read position corresponding to the cluster of oligonucleotides based on the adjusted signal as part of the server device(s) 102.

Though FIG. 1 illustrates the components of environment 100 communicating via the network 112, in some embodiments, the components of environment 100 communicate directly with each other, bypassing the network. For instance, and as previously mentioned, the user client device 108 can communicate directly with the sequencing device 114. Additionally, the user client device 108 can communicate directly with the cluster-aware-base-calling system 106, bypassing the network 112. Moreover, the cluster-aware-base-calling system 106 can access one or more databases housed on the server device(s) 102 or elsewhere in the environment 100.

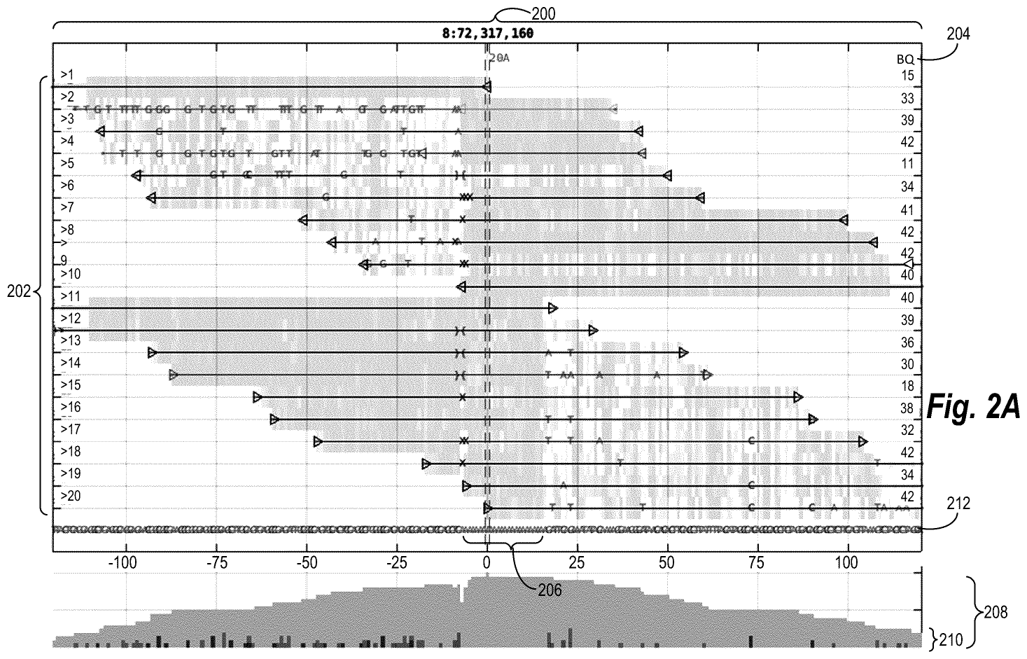

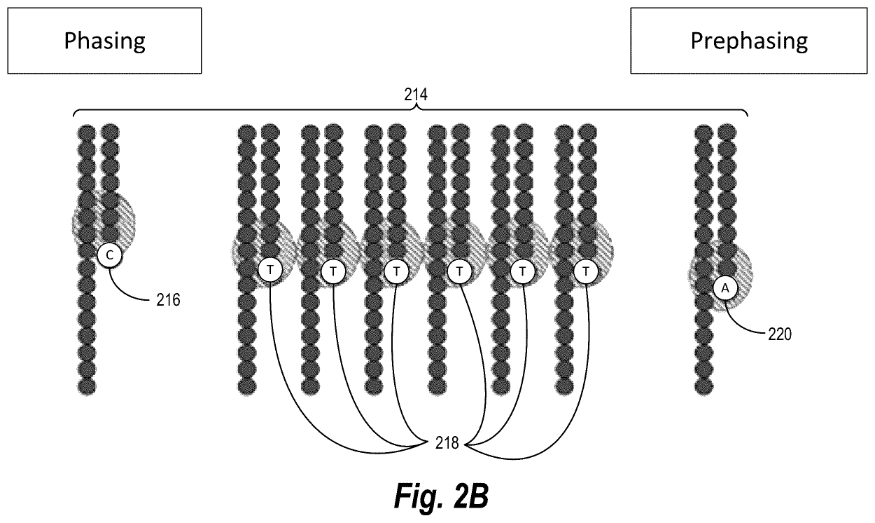

As previously mentioned, the cluster-aware-base-calling system 106 can determine a cluster-specific-phasing correction to correct a signal for estimated phasing and estimated pre-phasing. The following figures and discussion provide additional detail regarding how the cluster-aware-base-calling system 106 estimates the cluster-specific-phasing correction in accordance with some embodiments. In particular, FIG. 2A illustrates an example read pileup including several nucleotide-fragment reads that demonstrate the effects of phasing and pre-phasing by an error-inducing sequence in accordance with one or more embodiments. By contrast, FIG. 2B illustrates how phasing and pre-phasing occur at a molecular level in accordance with one or more embodiments.

As mentioned, FIG. 2A illustrates an example read pileup reflecting the effects of error-inducing sequences on base-call accuracy and secondary sequencing metrics in accordance with one or more embodiments. In particular, FIG. 2A illustrates a read pileup 200 comprising nucleotide-fragment reads 202 for a reference genome 212 with a homopolymer 206. FIG. 2A also depicts base quality 204, base depth 208, and error type counter 210 corresponding to the nucleotide-fragment reads 202 of the read pileup 200.

As mentioned, the read pileup 200 reflects data regarding several sequencing cycles. In particular, the base depth 208 reflects how many reads within the nucleotide-fragment reads 202 cover each base. For example, the base depth 208 includes light-gray bars that indicate a greater number of reads covering bases that have the most overlap between the forward and reverse nucleotide-fragment reads 202. To illustrate, bases in the center of the read pileup 200 correspond with the greatest number of reads.

As illustrated in FIG. 2A, the read pileup 200 includes the nucleotide-fragment reads 202. Generally, the nucleotide-fragment reads 202 indicate sequences of various DNA fragments within a genome. As mentioned previously, in some embodiments, the cluster-aware-base-calling system 106 can utilize the sequencing device 114 to generate the nucleotide-fragment reads 202. During such sequencing, the cluster-aware-base-calling system 106 can determine each of the nucleotide-fragment reads 202 based on the labeled nucleotide bases incorporated into the oligonucleotides of respective clusters. The cluster-aware-base-calling system 106 further aligns the nucleotide-fragment reads 202 along the reference genome 212 to determine nucleotide-base calls for the reference genome 212.

As further illustrated in FIG. 2A, the read pileup 200 indicates the read direction and errors for the nucleotide-fragment reads 202. For example, and as illustrated by the arrows at the ends of the nucleotide-fragment reads 202, the nucleotide-fragment reads 202 labeled 1-10 comprise labeled-nucleotide bases that are added by cycle in a reverse direction. The nucleotide-fragment reads 202 labeled 11-20 comprises labeled-nucleotide bases that are added by cycle in a forward direction. The vertical gray lines or shading overlapping the nucleotide-fragment reads 202 indicate correct nucleotide-base calls. More specifically, correct nucleotide-base calls match nucleotide bases of a reference genome. Letters within the nucleotide-fragment reads 202 indicate incorrect nucleotide-base calls that do not match bases from the reference genome 212.

As illustrated in FIG. 2A, the read pileup 200 includes the base quality 204. The base quality 204 reflects the base quality for each of the nucleotide-fragment reads 202. Generally, a greater occurrence of correct nucleotide-base calls corresponds with higher base quality, and incorrect nucleotide-base calls correspond with lower base quality. For example, in some embodiments, the base quality 204 reflects a Phred score (Q30) estimating the probability that a base call within one of the nucleotide-fragment reads 202 is wrong. By contrast, the error type counter 210 indicates the number of errors of each type of incorrect base call using a color-coded bar or grey-scale-shaded bar at various genomic coordinates. For example, in some embodiments, the error type counter 210 includes a color-coded bar chart that indicates the incorrect nucleotide-base call.

As the incorrect nucleotide-base calls indicate in FIG. 2A, the reference genome 212 contains an error-inducing sequence. In particular, the reference genome 212 contains the homopolymer 206. The homopolymer 206 comprises a sequence having consecutive A nucleotides. As shown in FIG. 2A, the number of incorrect nucleotide-base calls increases at various read positions following the homopolymer 206. For example, for nucleotide-fragment read 2, the number of errors increases for nucleotide bases after the homopolymer 206. Similarly, for nucleotide-fragment read 13, errors also increase after the homopolymer 206. But the incorrect nucleotide-base calls differ at the same read positions within nucleotide-fragment reads 1-10. Such error variance indicates an error-inducing sequence (here, the homopolymer 206) exhibits phasing or pre-phasing effects on the signals corresponding to the read positions following the error-inducing sequence.

As indicated in FIG. 2A, incorrect nucleotide-base calls follow an error-inducing sequence consistent with the direction of the nucleotide-fragment read. In particular, nucleotide-base calls for the nucleotide-fragment reads 202 are often accurate and correspond with high base quality before error-inducing sequences. Upon encountering an error-inducing sequence, SBS polymerases may slip or otherwise fail to accurately incorporate additional labeled nucleotide bases. To illustrate, and as previously mentioned, the nucleotide-fragment reads 1-10 are reverse reads while the nucleotide-fragment reads 11-20 are forward reads. As illustrated in FIG. 2A, the number of errors increase after the homopolymer 206 consistent with the direction of the nucleotide-fragment read. Accordingly, in some embodiments, the cluster-aware-base-calling system 106 determines that the read position follows the error-inducing sequence consistent with the direction of the nucleotide-fragment read.

As further depicted in FIG. 2A, the error type counter 210 indicates the location and magnitude of base-call errors within the nucleotide-fragment reads 202. As illustrated in FIG. 2A, the error type counter 210 also indicates the increased occurrence of base-call errors surrounding the homopolymer 206.

As depicted in FIG. 2A, an error-inducing sequence can cause phasing and pre-phasing effects in signals for clusters of oligonucleotides at read positions following the error-inducing sequence. As mentioned, FIG. 2B illustrates example oligonucleotides within a cluster to demonstrate phasing and pre-phasing in accordance with one or more embodiments. In particular, FIG. 2B illustrates oligonucleotides 214 within a particular cluster during a sequencing cycle. Generally, the labeled nucleotide bases 218 for the cycle comprise labeled nucleotide bases that fluoresce in response to a light signal during the cycle. For instance, labeled T nucleotide bases have been added to the majority of oligonucleotides for the given cycle illustrated in FIG. 2B.

FIG. 2B also illustrates phasing and pre-phasing. In an example of phasing, FIG. 2B illustrates a sequencing device incorporating, into an oligonucleotide, a labeled nucleotide base 216 (here, “C”) corresponding to a previous cycle instead of one of the labeled nucleotide bases 218 (here, “T”) corresponding to a current cycle. Accordingly, the labeled nucleotide base 216 for the previous cycle is accordingly incorporated one cycle late. In an example of pre-phasing, FIG. 2B illustrates the sequencing device incorporating, into a different oligonucleotide, a labeled nucleotide base 220 (here, “A”) corresponding to a subsequent cycle instead of one of the labeled nucleotide bases 218 (here, “T”) corresponding to the current cycle. Accordingly, the labeled nucleotide base 220 for a subsequent cycle is incorporated one cycle early.

As suggested by FIG. 2B, both phasing and pre-phasing impact the signal from labeled nucleotide bases within the cluster. In particular, instead of detecting a pure signal comprising light emitted by the labeled nucleotide bases 218 for the current cycle, the cluster-aware-base-calling system 106 detects a mixed signal including fluorescence from the labeled nucleotide base 216 for a previous cycle and the labeled nucleotide base 220 for a subsequent cycle. The following figures and paragraphs further describe how the cluster-aware-base-calling system 106 generates a cluster-specific-phasing correction to adjust the signal and account for a phased nucleotide base and a pre-phased nucleotide base.

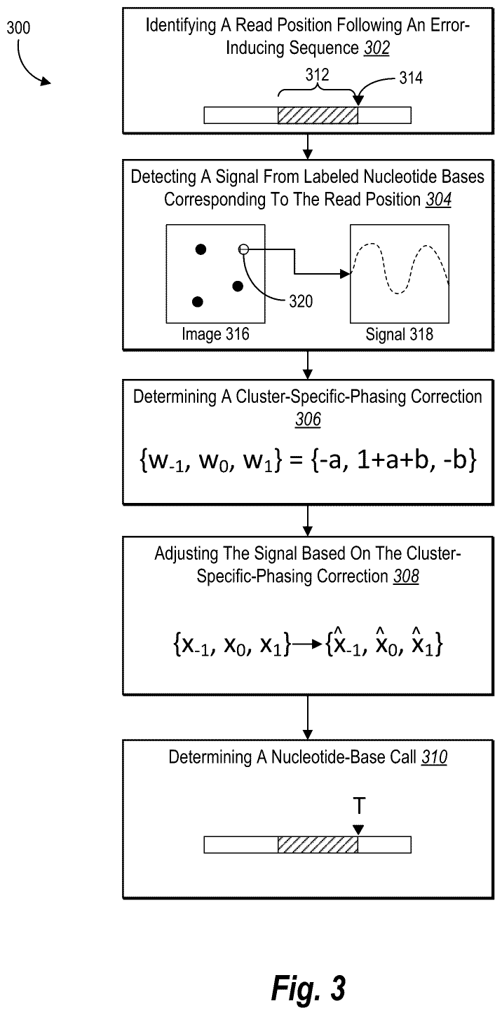

FIG. 3 provides an overview of the cluster-aware-base-calling system 106 generating a cluster-specific-phasing correction and adjusting a signal to determine an accurate nucleotide-base-call corresponding to a particular cluster. As an overview of FIG. 3, the cluster-aware-base-calling system 106 performs a series of acts 300 that includes an act 302 of identifying a read position following an error-inducing sequence, an act 304 of detecting a signal from labeled nucleotide bases corresponding to the read position, an act 306 of determining a cluster-specific-phasing correction, an act 308 of adjusting the signal based on the cluster-specific-phasing correction, and an act 310 of determining a nucleotide-base call.

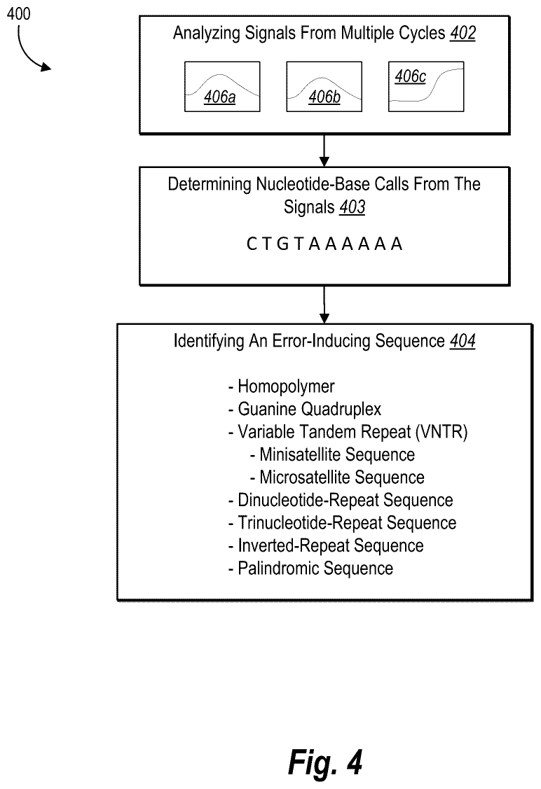

As just indicated, FIG. 3 illustrates the act 302 of identifying a read position following an error-inducing sequence. As mentioned, in some embodiments, the cluster-aware-base-calling system 106 limits the computing resources required to correct a signal for a cluster in part by limiting cluster-specific-phasing corrections to signals for read positions following identified error-inducing sequences. As illustrated in FIG. 3, in some embodiments, the cluster-aware-base-calling system 106 identifies an error-inducing sequence 312 by identifying a homopolymer, a guanine quadraplex, a VNTR, or other error-inducing sequence based on nucleotide-base calls for signals from previous cycles. In one example, the cluster-aware-base-calling system 106 analyzes signals from previous cycles and determines that the signals from a threshold number of previous cycles indicate the same nucleotide-base. The cluster-aware-base-calling system 106 thus determines the presence of a homopolymer, which is an error-inducing sequence. FIG. 4 and the corresponding discussion provide additional detail and examples of error-inducing sequences.

As part of the act 302, the cluster-aware-base-calling system 106 identifies a read position following an error-inducing sequence. As illustrated in FIG. 3, for instance, the cluster-aware-base-calling system 106 identifies a read position 314 following the error-inducing sequence 312. In some embodiments, the cluster-aware-base-calling system 106 identifies the read position 314 after an identified end of the error-inducing sequence 312. For example, if the error-inducing sequence 312 comprises a homopolymer having nucleotide bases emitting signals within a threshold similarity, the cluster-aware-base-calling system 106 can identify the read position 314 at a first position or second position where the labeled nucleotide bases emit a different signal. Additionally or alternatively, the cluster-aware-base-calling system 106 identifies one or more read positions (i) following the error-inducing sequence until a last position of the nucleotide-fragment read or (ii) within a threshold number of read positions following the error-inducing sequence 312 (e.g., within 200 or 300 nucleotide bases following an error-inducing sequence).

After identifying such a read position, the cluster-aware-base-calling system 106 performs the act 304 of detecting a signal from labeled nucleotide bases corresponding to the read position. In particular, when performing the act 304, the cluster-aware-base-calling system 106 detects a signal from labeled nucleotide bases within the cluster of oligonucleotides during a cycle corresponding to the read position. Accordingly, as part of performing the act 304, the cluster-aware-base-calling system 106 identifies a cycle corresponding to the read position 314 by identifying the cycle within which labeled nucleotide bases will be incorporated within the oligonucleotide at the read position 314. In one example, the cluster-aware-base-calling system 106 identifies a cycle immediately following or following within a threshold number (e.g., within 2 cycles from) previous cycles corresponding with the error-inducing sequence 312.

As further illustrated in FIG. 3, when performing the act 304, the cluster-aware-base-calling system 106 can capture an image 316 of a cluster 320. In some embodiments, the cluster-aware-base-calling system 106 captures the image 316 of at least one section of a nucleotide-sample slide utilizing a camera of a sequencing device. In this example, the image 316 portrays several clusters within a tile of a nucleotide-sample slide. In additional embodiments, the cluster-aware-base-calling system 106 captures one or more images of other parts of a nucleotide-sample slide, such as a sub-section, tile, channel, or other portions of a nucleotide-sample slide. As further shown, the image 316 portrays a signal 318 emitted from the cluster 320. The signal 318 comprises a light signal emitted from the labeled nucleotide bases incorporated within the cluster of oligonucleotides during the cycle.

After detecting such a signal from labeled nucleotide bases within a relevant cluster, the cluster-aware-base-calling system 106 performs the act 306 of determining a cluster-specific-phasing correction. In particular, when performing the act 306, the cluster-aware-base-calling system 106 determines, for the cluster of oligonucleotides, a cluster-specific-phasing correction to correct the signal for estimated phasing and estimated pre-phasing. More specifically, in some embodiments, the cluster-aware-base-calling system 106 determines (i) a cluster-specific-phasing coefficient corresponding to a nucleotide base for a previous cycle and (ii) a cluster-specific-pre-phasing coefficient corresponding to a nucleotide base for a subsequent cycle. For example, and as illustrated in FIG. 3, the coefficient a represents the cluster-specific-phasing coefficient, and the coefficient b represents the cluster-specific-pre-phasing coefficient. The cluster-aware-base-calling system 106 can further utilize the coefficients as part of an algorithm or function to determine the cluster-specific-phasing correction. For example, in some embodiments, the cluster-aware-base-calling system 106 utilizes the cluster-specific-phasing coefficient and the cluster-specific-pre-phasing coefficient within a Finite Impulse Response (FIR) filter.

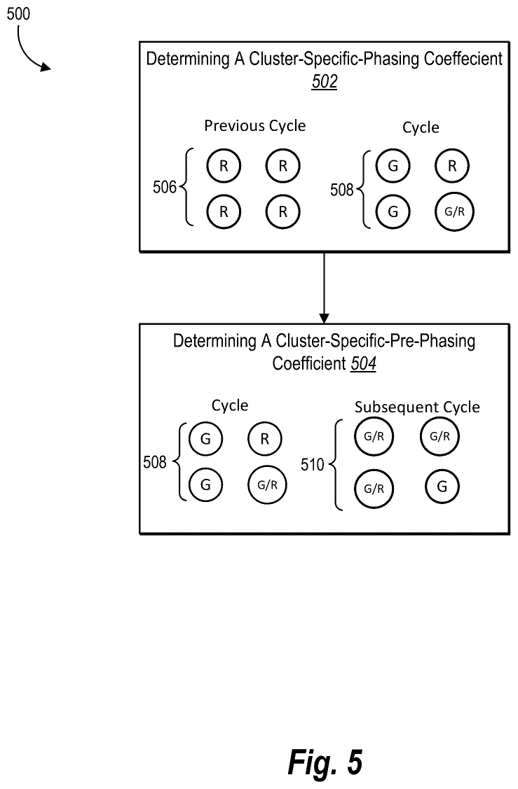

While FIG. 3 illustrates determining a single cluster-specific-phasing coefficient and a single cluster-specific-pre-phasing coefficient, in some embodiments, the cluster-aware-base-calling system 106 determines multiple additional coefficients corresponding to more previous cycles (e.g., two, three, four, etc. previous cycles) and/or more subsequent cycles (e.g., two, three, four, etc. subsequent cycles). FIG. 5 and the corresponding paragraphs further detail how the cluster-aware-base-calling system 106 determines the cluster-specific-phasing coefficient a and the cluster-specific-pre-phasing coefficient b in accordance with one or more embodiments.

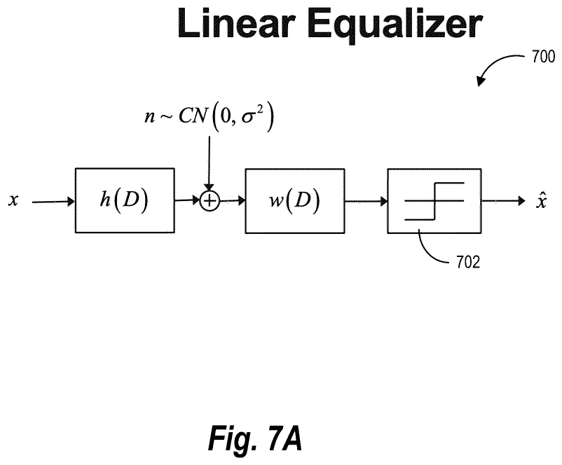

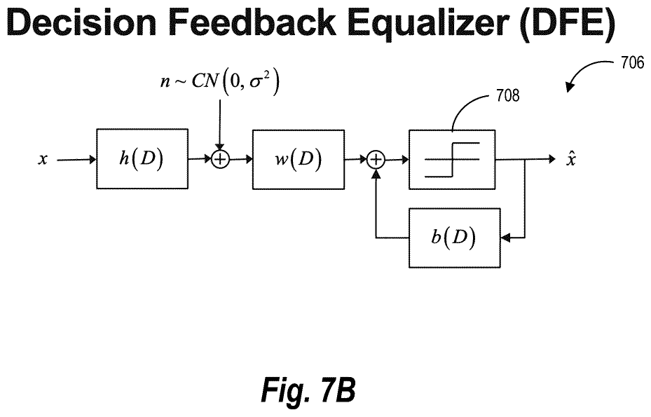

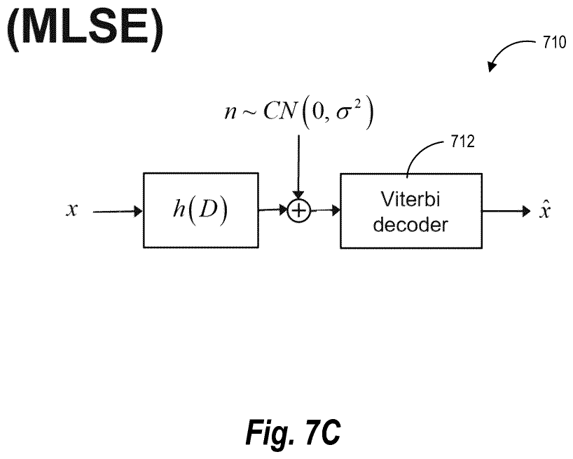

The cluster-aware-base-calling system 106 can utilize a number of models as part of performing the act 306 of determining a cluster-specific-phasing correction. For example, the cluster-aware-base-calling system 106 can utilize a Linear Equalizer (LE), Decision Feedback Equalizer (DFE), or a Maximum Likelihood Sequence Estimator (MLSE) to determine the cluster-specific-phasing coefficient and the cluster-specific-pre-phasing coefficient. FIGS. 7A-7C and the accompanying discussion provide additional detail regarding each of these models.

In some embodiments, as part of performing the act 306, the cluster-aware-base-calling system 106 utilizes the cluster-specific-phasing coefficient a and the cluster-specific-pre-phasing coefficient b to determine weights corresponding to a previous cycle (w₋₁), the current cycle (w₀), and a subsequent cycle (w₁). In some embodiments, the weights represent equalizer coefficients that the cluster-aware-base-calling system 106 utilizes to adjust signals. While FIG. 3 illustrates a window of three weights corresponding to a previous cycle, the current cycle, and a subsequent cycle, the cluster-aware-base-calling system 106 can generate more weights as indicated above. For instance, the cluster-aware-base-calling system 106 can generate five weights. To illustrate, of the five weights, the cluster-aware-base-calling system 106 determines weights corresponding to a cycle preceding the previous cycle (w₋₂), the previous cycle (w₋₁), the current cycle (w₀), the subsequent cycle (w₁), and a cycle following the subsequent cycle (w₂). The cluster-aware-base-calling system 106 can accordingly expand the number of identified weights to seven, nine, or any relevant window.

After determining a cluster-specific-phasing correction, the cluster-aware-base-calling system 106 performs an act 308 of adjusting the signal based on the cluster-specific-phasing correction. Generally, the cluster-aware-base-calling system 106 adjusts the signal based on the cluster-specific-phasing coefficient (a) and the cluster-specific-pre-phasing coefficient (b). In some embodiments, the cluster-aware-base-calling system 106 performs the act 308 by applying the weights described above to the signal from the cluster of oligonucleotides. For example, FIG. 3 represents the signals for the previous cycle, cycle, and subsequent cycle as {x₋₁, x₀, x₁}. The cluster-aware-base-calling system 106 applies the weights for the previous cycle, current cycle, and subsequent cycle {w₋₁, w₀, w₁} to generate adjusted signals for the previous cycle, cycle, and subsequent cycle {\hat{x}₋₁, \hat{x}₀, \hat{x}₁}. In some embodiments, the cluster-aware-base-calling system 106 generates adjusted signals for additional cycles based on the number of weights determined in the previous step.

After adjusting the signal, the cluster-aware-base-calling system 106 performs an act 310 of determining a nucleotide-base call. In particular, when performing the act 310, the cluster-aware-base-calling system 106 determines a nucleotide-base call for the read position corresponding to the cluster of oligonucleotides based on the adjusted signal. For example, and as illustrated in FIG. 3, the cluster-aware-base-calling system 106 determines that the identity of the nucleotide base at the read position 314 is a thymine (T) based on the adjusted signal. Generally, the cluster-aware-base-calling system 106 can utilize the sequencing system 104 to generate nucleotide-base calls indicating the identify of nucleotide bases within a cluster to determine a nucleotide-fragment read. The cluster-aware-base-calling system 106 can further align the nucleotide-fragment reads resulting from the analysis of adjusted signals to indicate the sequence of a sample genome of other nucleic-acid polymer.

While FIG. 3 depicts the cluster-aware-base-calling system 106 determining a cluster-specific-phasing coefficient and a cluster-specific-pre-phasing coefficient—and adjusting a signal based on such coefficients—for a signal from a given cluster at or during a sequencing cycle, in some embodiments, the cluster-aware-base-calling system 106 can determine and re-determine such coefficients for a signal from a given cluster as sequencing cycles continue. For instance, in some embodiments, the cluster-aware-base-calling system 106 can determine a cluster-specific-phasing coefficient and a cluster-specific-pre-phasing coefficient (and corresponding weights) for a given cluster of oligonucleotides at on sequencing cycle and then determine an updated cluster-specific-phasing coefficient and an updated cluster-specific-pre-phasing coefficient (and corresponding weights) for the given cluster of oligonucleotides at a subsequent sequencing cycle, and so on and so forth for each subsequent cycle. Accordingly, the cluster-aware-base-calling system 106 re-determines and changes cluster-specific-phasing coefficients and cluster-specific-pre-phasing coefficients for a given cluster of oligonucleotides over the course of determining nucleotide-base calls for a nucleotide-fragment read corresponding to the given cluster.

FIG. 3 provides an overview of acts performed by the cluster-aware-base-calling system 106 as part of determining a nucleotide-base call from a signal adjusted for estimated phasing and pre-phasing in accordance with one or more embodiments. FIG. 4 illustrates a series of acts performed by the cluster-aware-base-calling system 106 to identify an error-inducing sequence in accordance with one or more embodiments. Generally, the cluster-aware-base-calling system 106 selectively determines a cluster-specific-phasing correction and adjusts signals from particular cycles following error-inducing sequences according to the cluster-specific-phasing correction. As depicted by a series of acts 400 in FIG. 4, the cluster-aware-base-calling system 106 identifies an error-inducing sequence by performing an act 402 of analyzing signals from multiple cycles, an act 403 of determining nucleotide-base calls from the signals, and an act 404 of identifying an error-inducing sequence.

As illustrated in FIG. 4, the cluster-aware-base-calling system 106 performs the act 402 of analyzing signals from multiple cycles. Generally, the cluster-aware-base-calling system 106 detects signals from labeled nucleotide bases from a cluster by taking one or more images of the cluster. More specifically, the cluster-aware-base-calling system 106 captures one or more images of a section of a nucleotide-sample-slide (e.g., a tile of a flow cell) containing multiple clusters. The images capture signals emitted from the cluster. The cluster-aware-base-calling system 106 analyzes the images to detect signals 406a-406c. The signals 406a-406c comprise signals emitted from labeled nucleotide bases within the cluster for different cycles. For instance, the cluster-aware-base-calling system 106 records the signal 406a for a first cycle, the signal 406b for a second cycle, and the signal 406c for a third cycle.

In some embodiments, the signals 406a-406c are derived from images obtained from different detection channels. For example, the signals 406a-406c can be generated based on resulting images from 2-channel or 4-channel sequencing. Each nucleotide base is associated with a different signal. To illustrate, in 2-channel SBS, green clusters correspond with C nucleotide bases, red clusters correspond with T nucleotide bases, clusters observed in both red and green are flagged as A nucleotide bases, and unlabeled clusters correspond with G nucleotide bases. By contrast, in one or more embodiments, the cluster-aware-base-calling system 106 detects the signals from a single detection channel. For example, the signals 406a-406c are generated based on images obtained from 1-channel sequencing.

In some embodiments, as part of performing the act 402 of analyzing signals from multiple cycles, the cluster-aware-base-calling system 106 adjusts the signals 406a-406c for phasing/phrasing and noise. In particular, the cluster-aware-base-calling system 106 can determine a cluster-specific-phasing correction to correct the signals 406a-406c for estimated phasing and/or estimated pre-phasing. In one example, the cluster-aware-base-calling system 106 further analyzes signals from multiple cycles by adjusting the signals 406a-406c to reduce noise. For example, in some embodiments, the cluster-aware-base-calling system 106 utilizes de-noisers or algorithms for removing noise. Indeed, in some cases, noise is part of a signal and comprises signal variation that leads to (or reflects) a distribution in an observed population. The signal variation can come from chemical or physical properties of components or contents of a nucleotide-sample slide (e.g., a flow cell) or of a sequencing device, such as signal variation attributable to oligonucleotide length, phasing or pre-phasing, or a position of a cluster of oligonucleotides with respect to a camera or other sensor's field of view. In addition to removing noise, the cluster-aware-base-calling system 106 can further refine the signals 406a-406c to improve other metrics. For example, in some embodiments, the cluster-aware-base-calling system 106 adjusts the signals 406a-406c based on offset and a scaling factor corresponding to intensity values of the signals 406a-406c.

Furthermore, as part of performing the act 402 of analyzing signals from multiple cycles, the cluster-aware-base-calling system 106 compares intensity values for the adjusted signals with sets of intensity-value boundaries. Generally, intensity-value boundaries refer to decision boundaries used in generating a nucleotide-base call for a signal. In particular, intensity-value boundaries can refer to decision boundaries that classify a nucleotide base based on one or more intensity values of the signal. To illustrate, intensity-value boundaries can define or otherwise indicate the boundaries of a nucleotide cloud corresponding to each of the nucleotide bases. In particular, the cluster-aware-base-calling system 106 identifies sets of intensity-value boundaries corresponding to each possible nucleotide base (e.g., A, T, C, or G). In some embodiments, the cluster-aware-base-calling system 106 discards an adjusted signal having intensity values outside of one of the sets of intensity-value boundaries. For example, based on determining that an adjusted signal for a cluster has intensity values outside of one of the sets of intensity-value boundaries, the cluster-aware-base-calling system 106 determines to not generate a nucleotide-base call for the cluster.

As further illustrated in FIG. 4, the series of acts 400 includes the act 403 of determining nucleotide-base calls from the signals. In particular, the cluster-aware-base-calling system 106 can generate a nucleotide-base call for a signal utilizing one of the sets of intensity-value boundaries. In particular, the cluster-aware-base-calling system 106 can generate the nucleotide-base call utilizing the sets of intensity-value boundaries. Generally, based on determining a correlation between a set of intensity-value boundaries and the signal 406a, the cluster-aware-base-calling system 106 determines a nucleotide-base call for the cycle corresponding to an adjusted version of the signal 406a (i.e., an adjusted signal). For example, based on determining that intensity values corresponding to the adjusted version of the signal 406a (i.e., an adjusted signal) fall within a set of intensity-value boundaries corresponding to an A nucleotide base, the cluster-aware-base-calling system 106 determines an A nucleotide-base call.

In some embodiments, the cluster-aware-base-calling system 106 discards signal data after determining nucleotide-base calls. To reduce the storage load required to estimate cluster-specific-phasing corrections, the cluster-aware-base-calling system 106 can periodically delete or discard signal data. For example, in some embodiments, the cluster-aware-base-calling system 106 discards signal data within a threshold number of cycles. For example, the cluster-aware-base-calling system 106 can delete signal data within a threshold number of cycles (e.g., 3, 5, 10, etc.) of determining a nucleotide-base call for a particular cycle. As mentioned previously, the cluster-aware-base-calling system 106 selectively corrects signals for a cycle corresponding to a read position following an error-inducing sequence. Accordingly, in some cases, the cluster-aware-base-calling system 106 delete signal data for cycles unaffected by error-inducing sequences. In some embodiments, for a given cluster, the cluster-aware-base-calling system 106 identifies cycles unaffected by error-inducing sequences and discards the corresponding signal data. For example, the cluster-aware-base-calling system 106 can determine that nucleotide-base calls for previous cycles do not indicate an identifiable error-inducing sequence. Based on this determination, the cluster-aware-base-calling system 106 discards signaling data for the cycle.

As further illustrated in FIG. 4, the cluster-aware-base-calling system 106 repeats the act 403 for multiple cycles. In particular, the cluster-aware-base-calling system 106 determines nucleotide-base calls for the signals from multiple cycles. The resulting sequence of nucleotide-base calls at each cycle for the cluster becomes a nucleotide-fragment read for the cluster. For example, and as illustrated in FIG. 4, the cluster-aware-base-calling system 106 generates a nucleotide-fragment read with the sequence “CTGTAAAAAA.”

As further illustrated in FIG. 4, the cluster-aware-base-calling system 106 performs the act 404 of identifying an error-inducing sequence. Generally, the cluster-aware-base-calling system 106 analyzes the sequence of nucleotide bases (corresponding to preceding cycles) from a nucleotide-fragment read to detect the presence of an error-inducing sequence. For instance, after determining a particular nucleotide-base call for a particular cycle, the cluster-aware-base-calling system 106 can compare a sequence of nucleotide-base calls from a growing nucleotide-fragment read to a database of possible error-inducing sequences. By using such a database of error-inducing sequences, the cluster-aware-base-calling system 106 can analyze the sequence of nucleotide-base calls to determine whether the nucleotide-fragment read includes an error-inducing sequence. When the sequence of nucleotide-base calls from such a nucleotide-fragment read matches (or is within a threshold number of nucleotide bases from) a particular error-inducing sequence, the cluster-aware-base-calling system 106 identifies the error-inducing sequence within the nucleotide-fragment read.

Generally, error-inducing sequences comprise sequences of one or more repeated nucleotide bases or sequence motifs. Sequence motifs can comprise nucleotide patterns that occur within a genome. In some examples, sequence motifs are related to a biological function. FIG. 4 illustrates a number of example error-inducing sequences in accordance with one or more embodiments. The following paragraphs describe various examples of error-inducing sequences identified by the cluster-aware-base-calling system 106. In some embodiments, a sequence recognition model identifies a trigger for an error-inducing sequence. For example, a sequence recognition model can comprise a machine learning model trained to identify or predict nucleotide base sequences that cause base-calling errors. Additionally, or alternatively, error-inducing sequences are identifiable based on the base count of a block or group of bases within a sequence.

As illustrated in FIG. 4, a homopolymer can be an error-inducing sequence. Generally, homopolymers comprise polymers consisting of or comprising identical monomer units. In particular, a homopolymer comprises a sequence having a single repeating nucleotide base. For example, a homopolymer can include a segment of fifteen or more repeating A nucleotides. Homopolymers often induce errors by causing polymerase slippage during clustering. Polymerase slippage occurs when a polymerase temporarily dissociates from an oligonucleotide and re-attaches at a different location. Such polymerase slippage often generates filaments of heterogenous length, which manifests as acute phasing or pre-phasing errors downstream. Homopolymers can comprise a repeated sequence of any nucleotide base, including homopolymers of A, T, G, or C. In some embodiments, near-homopolymers are also considered error-inducing sequences. In particular, near-homopolymers comprise polymers where every monomer, excepting a few, is the same. For example, a near-homopolymer can comprise a chain of repeating bases (e.g., 20) interrupted by a single different base.

Another example of an error-inducing sequence illustrated in FIG. 4 includes a guanine quadruplex (G-quadruplex). G-quadruplexes are stable secondary structures formed by sequences that are rich in guanine. In particular, G-quadruplexes form intra-strand secondary structures on a template oligonucleotide during SBS. G-quadruplexes can induce errors in SBS by blocking SBS polymerase. More specifically, polymerases that are washed off after a sequencing cycle are often less efficient at re-attaching, causing catastrophic phasing. The cluster-aware-base-calling system 106 may identify a G-quadruplex by identifying sequences rich in guanine. In some embodiments, the cluster-aware-base-calling system 106 can computationally predict G-quadruplex sequence motifs. For example, the cluster-aware-base-calling system 106 can utilize a machine learning model such as a sequence-based computational model to predict the formation of G-quadruplexes.

Some error-inducing sequences, such as G-quadruplexes, are more difficult to identify than other error-inducing sequences including homopolymers. For example, the cluster-aware-base-calling system 106 may erroneously detect the presence of a G-quadruplex and accordingly proceed to determining a cluster-specific phasing correction. This type of premature determination does not negatively impact performance but consumes additional resources. In some embodiments, the cluster-aware-base-calling system 106 does not determine a cluster-specific-phasing correction unless the error-inducing sequence is an easily identifiable nucleotide sequence, such as homopolymers and near-homopolymers.

As further illustrated in FIG. 4, variable tandem repeats (VNTRs) are another example of error-inducing sequences. A VNTR can comprise a location in a genome where a short nucleotide sequence (20-100 base pairs) is organized as a tandem repeat. For example, a VNTR can comprise a sequence made up of six repeating AGTCGGTAAG sequences or various other numbers of repeating subsequences. VNTRs may cause errors in SBS by causing polymerase slippage leading to downstream phasing and pre-phasing.

Other examples of VNTRs include minisatellite sequences and microsatellite sequences. Minisatellite sequences refer to tracts of repetitive DNA in which certain DNA motifs (ranging in length from 10-60 base pairs) are typically repeated 5-50 times. Microsatellite sequences are tracts of repetitive DNA in which certain DNA motifs (ranging in length from one to six or more base pairs) are typically repeated 5-50 times.

As further illustrated in FIG. 4, error-inducing sequences can also comprise dinucleotide-repeat sequences and trinucleotide repeat sequences. Dinucleotide-repeat sequences occur when exactly two nucleotides are repeated. An ATATAT sequence is an example of a dinucleotide-repeat sequence. Similarly, trinucleotide-repeat sequences occur when exactly three nucleotides are repeated. For instance, the DNA sequence CAGCAGCAGCAG contains four CAG repeats. Dinucleotide- and trinucleotide-repeat sequences negatively impact SBS by causing polymerase slippage. Additionally, in some examples, dinucleotide- and trinucleotide-repeat sequences can also negatively impact PCR preparation steps of SBS.

Another example of an error-inducing sequence illustrated in FIG. 4 is an inverted-repeat sequence. An inverted-repeat sequence comprises a single stranded sequence of nucleotides followed downstream by its reverse complement. The intervening sequence of nucleotides between the initial sequence and the reverse complement can be any length including zero. For example, TTACGnnnnCGTAA is an inverted-repeat sequence. Inverted-repeat sequences can often cause inter-strand hairpins or intra-strand hybridization. The resulting secondary structure often block SBS polymerases from reattaching to the oligonucleotide during SBS.

Palindromic sequences represent another example of error-inducing sequence identifiable by the cluster-aware-base-calling system 106. Palindromic sequences comprise a first run of nucleotide bases followed by a second run of complementary bases in reverse order. GGATCC is an example of a palindromic sequence. Palindromic sequences can be problematic during SBS because they cause intra-stand and inter-strand hybridization within a cluster. For example, a palindromic sequence can cause hybridization within the motif itself. Palindromic sequences can also cause inter-strand hybridization in which a sequence on one oligonucleotide hybridizes with the sequence on a second oligonucleotide. Both forms of interactions block polymerases during SBS.

In some embodiments, the cluster-aware-base-calling system 106 identifies a direction-specific sequence motif. In particular, the cluster-aware-base-calling system 106 can flag a sequence motif as an error-inducing sequence based on determining that the sequence motif is in a particular direction. The cluster-aware-base-calling system 106 can determine that the same sequence motif in the opposite direction does not comprise an error-inducing sequence. In one example, a G-quadruplex on a forward strand can create an intra-strand secondary structure during SBS and negatively impact sequencing reads. In contrast, the reverse or complementary strand of the G-quadruplex usually do not create intra-strand secondary structures (unless the reverse direction also includes a G-quadruplex). Other error-inducing sequences that tend to form intra-strand secondary structures can also be direction-specific sequence motifs.

FIG. 4 and the accompanying discussion above describe the cluster-aware-base-calling system 106 identifying an error-inducing sequence within a nucleotide-fragment read in accordance with one or more embodiments. As described previously, the cluster-aware-base-calling system 106 also identifies a read position following an error-inducing sequence. The cluster-aware-base-calling system 106 further processes a signal from labeled nucleotide bases during a cycle corresponding to the read position. As part of processing the signal, the cluster-aware-base-calling system 106 determines a cluster-specific-phasing correction to correct the signal. In particular, the cluster-aware-base-calling system 106 can determine the cluster-specific-phasing correction based on a cluster-specific-phasing coefficient and a cluster-specific-pre-phasing coefficient. FIG. 5 and the corresponding paragraphs describe a series of acts 500 for determining a cluster-specific-phasing coefficient and determining a cluster-specific-pre-phasing coefficient in accordance with one or more embodiments.

As shown in FIG. 5, the cluster-aware-base-calling system 106 performs an act 502 of determining a cluster-specific-phasing coefficient. In particular, as part of the act 502, the cluster-aware-base-calling system 106 determines, for the cluster of oligonucleotides, a cluster-specific-phasing coefficient corresponding to a nucleotide base for a previous cycle.

FIG. 5 illustrates signals emitted from labeled nucleotide bases within a cluster of oligonucleotides. For example, FIG. 5 illustrates current-cycle signals 508 from labeled nucleotide bases within a single cluster for the cycle and previous cycle signals 506 from labeled nucleotide bases within the cluster for a previous cycle. Along with other labeled nucleotide bases incorporated in oligonucleotides of the cluster (not shown), the cluster emits a collective signal captured by an image. For ease of explanation, this disclosure refers to previous cycle signals 506, current-cycle signals 508, and subsequent-cycle signals 510 as the collection of signals that make up a collective signal for a cluster for a given cycle. As shown, each circle represents a signal emitted by a single labeled nucleotide base within a cluster. As illustrated, the current-cycle signals 508 include two labeled nucleotide bases emitting green light, on labeled nucleotide base emitting red light, and one labeled nucleotide base emitting both green and red.

In some embodiments, the cluster-aware-base-calling system 106 determines a cluster-specific-phasing coefficient corresponding to a nucleotide base for a previous cycle that immediately precedes a current cycle. As mentioned, phasing occurs when one or more oligonucleotides within a cluster fall behind incorporating nucleotide bases. For instance, and as illustrated in FIG. 5, the cluster-aware-base-calling system 106 identifies previous cycle signals 506. The previous cycle signals 506 indicate that labeled nucleotides added to oligonucleotides within the cluster during the previous cycle emit red signals. The current-cycle signals 508 indicate that phasing has occurred during the cycle. More specifically, the current-cycle signals 508 include one labeled nucleotide base emitting red light, which corresponds with the red light for the previous cycle signals 506. As explained further below, the cluster-aware-base-calling system 106 determines a cluster-specific-phasing coefficient corresponding to the nucleotide base for the previous cycle.

As further illustrated in FIG. 5, the cluster-aware-base-calling system 106 also performs the act 504 of determining a cluster-specific-pre-phasing coefficient. In particular, the cluster-aware-base-calling system 106 determines, for a cluster of oligonucleotides, a cluster-specific-pre-phasing coefficient corresponding to a nucleotide base for a subsequent cycle immediately following the cycle. As mentioned, pre-phasing occurs when one or more oligonucleotides incorporate a nucleotide base one or more cycles early. As illustrated in FIG. 5, the current-cycle signals 508 includes a labeled nucleotide base emitting a combination of green and red light. The green and red (G/R) light emitted by the labeled nucleotide within the cluster corresponds to the G/R-labeled nucleotides from subsequent-cycle signals 510. As explained further below, as part of performing the act 504, the cluster-aware-base-calling system 106 determines a cluster-specific-pre-phasing coefficient corresponding to the G/R nucleotide base from the subsequent cycle.

In some embodiments, the cluster-aware-base-calling system 106 determines the cluster-specific-pre-phasing coefficient and the cluster-specific-phasing coefficient based on an input signal, a desired output signal, and various parameters. In particular, in one or more implementations in which the cluster-aware-base-calling system 106 utilizes a 3-tap linear equalizer, the cluster-aware-base-calling system 106 generates a cluster-specific-pre-phasing coefficient and a cluster-specific-phasing coefficient for a 3-tap linear equalizer based on an input signal (v), a desired output signal (d), and parameters including the mean (μ) and standard deviation (a) of the distributions. Generally, the cluster-aware-base-calling system 106 utilizes decision directed adaptation. In particular, the cluster-aware-base-calling system 106 sets the desired output signal (d) to the centers of clouds of base calls and uses the desired output signal (d) to update the parameters including the mean (μ) and standard deviation (a) of the distributions. Specific examples of how the cluster-aware-base-calling system 106 determines the cluster-specific-phasing coefficient and the cluster-specific-pre-phasing coefficient are provided below in the paragraphs accompanying FIG. 7A.

While FIG. 5 illustrates the cluster-aware-base-calling system 106 determining a cluster-specific-phasing coefficient and a cluster-specific-pre-phasing coefficient, in some embodiments, the cluster-aware-base-calling system 106 determines additional cluster-specific-phasing coefficients and additional cluster-specific-pre-phasing coefficients. Phasing can refer to instances where nucleotide bases are added one cycle late, and pre-phasing can refer to instances where nucleotide bases are added one cycle early. However, phasing and pre-phasing can also refer to nucleotide bases added two or more cycles late and two or more cycles early, respectively. Accordingly, in some embodiments, the cluster-aware-base-calling system 106 determines an additional cluster-specific-phasing coefficient corresponding to an additional nucleotide base for an additional previous cycle (i.e., two cycles before the cycle). The cluster-aware-base-calling system 106 can also determine an additional cluster-specific-pre-phasing coefficient corresponding to an additional nucleotide base for an additional subsequent cycle (i.e., two cycles after the cycle).

The cluster-aware-base-calling system 106 can also determine sets of cluster-specific-phasing coefficients corresponding to a set of nucleotide bases for a set of previous cycles immediately preceding the cycle. Such a set of previous cycles can include any number of preceding cycles. Similarly, the cluster-aware-base-calling system 106 can also determine sets of cluster-specific-pre-phasing coefficients corresponding to a set of subsequent cycles immediately following the cycle. Such a set of subsequent cycles can include any number of following cycles.

In some embodiments, the cluster-aware-base-calling system 106 analyzes signals from asymmetrical sets of previous cycles and sets of subsequent cycles. For example, the cluster-aware-base-calling system 106 can (i) process a signal and determine a cluster-specific-phasing coefficient for a single preceding cycle and (ii) process a plurality of signals and determine cluster-specific-pre-phasing coefficients for a plurality of subsequent cycles (e.g., two or three subsequent cycles). As a further example, the cluster-aware-base-calling system 106 can (i) process a plurality of signals and determine cluster-specific-phasing coefficients for a plurality of preceding cycles (e.g., two or three previous cycles) and (ii) process a single signal and determine a cluster-specific-pre-phasing coefficient for a single subsequent cycle. Additionally, or alternatively, the cluster-aware-base-calling system 106 can process signals from non-continuous cycles. To illustrate, the cluster-aware-base-calling system 106 can analyze and determine a cluster-specific coefficient for a signal from a cycle preceding the previous cycle, the current cycle, and a subsequent cycle. In this example, the cluster-aware-base-calling system 106 determines not to analyze a signal from the previous cycle, but could select another non-contiguous cycle before or after a current cycle.

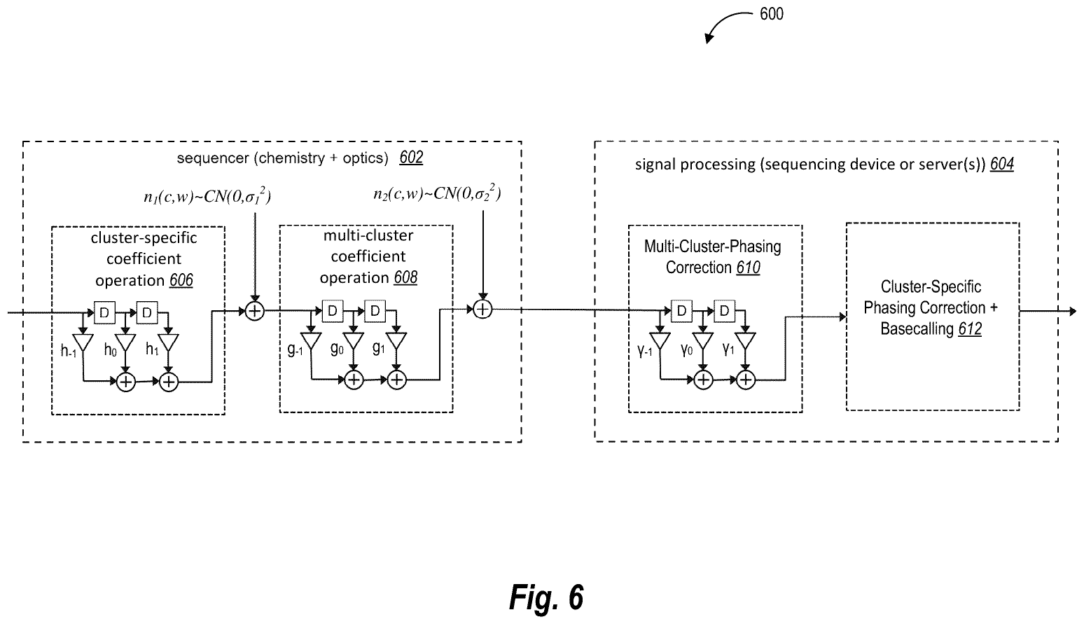

As described, FIG. 5 illustrates the cluster-aware-base-calling system 106 determining a cluster-specific-phasing coefficient and a cluster-specific-pre-phasing coefficient as part of determining a cluster-specific phasing correction in accordance with one or more embodiments. In some embodiments, the cluster-aware-base-calling system 106 determines cluster-specific-phasing corrections together with various algorithms. FIG. 6 illustrates an example phasing model for determining phasing corrections in accordance with one or more embodiments. Generally, the cluster-aware-base-calling system 106 can determine a cluster-specific-phasing correction to correct a signal from a cluster of oligonucleotides—as well as multi-cluster-phasing corrections to correct the signal from the cluster and signals from a set of clusters. FIG. 6 illustrates cluster-specific coefficient operation 606 and multi-cluster coefficient operation 608 modeled as two convolution operations in series.

In particular, FIG. 6 illustrates a phasing model 600 for estimating various coefficients as part of generating a cluster-specific-phasing correction and a multi-cluster-phasing correction. The phasing model 600 includes operations occurring on a sequencer 602 or other sequencing machine as well as operations occurring during signal processing 604. For example, in some embodiments, the cluster-aware-base-calling system 106 performs the cluster-specific coefficient operation 606 to estimate cluster-specific-phasing coefficients and the multi-cluster coefficient operation 608 to estimate multi-cluster-phasing coefficients. The cluster-aware-base-calling system 106 can further utilize the cluster-specific-phasing coefficients and the multi-cluster-phasing coefficients as part of the signal processing 604. More specifically, the cluster-aware-base-calling system 106 performs multi-cluster-phasing correction 610 to adjust a signal based on the multi-cluster-phasing coefficients. Furthermore, the cluster-aware-base-calling system 106 performs cluster-specific phasing correction and base calling 612 to adjust the signal based on cluster-specific-phasing coefficients and generate a nucleotide-base call based on the adjusted signal.

The phasing model 600 can comprise a real-time (or near real-time) computing architecture or a buffered computing architecture. Generally, by utilizing a real-time computing architecture, the cluster-aware-base-calling system 106 performs all operations illustrated in FIG. 6 utilizing a processor of the sequencer 602 (e.g., the sequencing device 114). In contrast, the cluster-aware-base-calling system 106 may also employ a buffered computing architecture that involves both a sequencing machine and one or more servers (e.g., the server device(s) 102). In one example, the cluster-aware-base-calling system 106 performs the signal processing 604 at one or more server devices while performing the cluster-specific coefficient operation 606 and the multi-cluster coefficient operation 608 at the sequencer 602. More specifically, the cluster-aware-base-calling system 106 can perform (i) the multi-cluster-phasing correction 610 and (ii) the cluster-specific phasing correction and base calling 612 at the processor of a server device.

Generally, and as previously described, phasing and pre-phasing refer to phenomenon where a fraction of oligonucleotides in a cluster shift forward or backward by incorporating nucleotide bases corresponding to one or more previous or subsequent cycles, respectively. The cluster-aware-base-calling system 106 can produce a corrected signal (the output signal y) based on a convolution of a signal for a cluster (input signal x) and cluster-specific-phasing coefficient (input coefficients h). More particularly, the cluster-specific-phasing coefficient (h) includes both the cluster-specific-pre-phasing coefficient and the cluster-specific-phasing coefficient. The corrected signal can be modeled as a convolution operation y_{c}=Σᵢhᵢx_{c-i}, which is written as y=x*h. Assuming no signal decay, the cluster-specific coefficient h is constrained by Σᵢhᵢ=1, hᵢ≥0. In signal processing and communication systems literature, it is common to use D-transform notation, where D^{k} indicates a delay of k cycles: h(D)= . . . +h₋₂D⁻²+h₋₁D⁻¹+h₀+h₁D+h₂D²+ . . . . As written, h₋₂D⁻²+h₋₁D⁻¹ represents phasing coefficients corresponding to nucleotide bases two and one cycles previous to the current cycle. h₁D+h₂D² represents pre-phasing coefficients corresponding to nucleotide bases one and two cycles following the current cycle.

As illustrated in FIG. 6, the cluster-aware-base-calling system 106 performs the cluster-specific coefficient operation 606 to determine the cluster-specific-phasing coefficient and the cluster-specific-pre-phasing coefficient for each cluster with read positions following an error-inducing sequence. To illustrate, the cluster-aware-base-calling system 106 determines various cluster-specific-phasing coefficients (h) corresponding to a previous cycle (h₋₁), a current cycle (h₀), and a subsequent cycle (h₁). The cluster-specific-phasing coefficients vary independently across clusters and may not be determined for some clusters (e.g., at read positions preceding or within an error-inducing sequence). Most clusters unaffected by estimated phasing or pre-phasing have the values h=[0 1 0]. However, the cluster-aware-base-calling system 106 can determine that the cluster-specific-phasing coefficients change randomly and abruptly after error-inducing sequences, such as homopolymers. In some embodiments, the cluster-specific-phasing coefficients sum to unity and are non-negative as represented by the function Σᵢhᵢ(c)=1,hᵢ≥0.

As further illustrated in FIG. 6, the cluster-aware-base-calling system 106 performs the multi-cluster coefficient operation 608 to determine a multi-cluster-phasing coefficient. The cluster-aware-base-calling system 106 can utilize the multi-cluster-phasing coefficient across clusters in a particular section of a nucleotide-sample slide (e.g., tile of a flow cell). The multi-cluster-phasing coefficient values can change gradually from cycle to cycle. These values are simpler to estimate accurately than cluster-specific-phasing coefficients because the statistics can be averaged across millions of clusters.

As shown in FIG. 6, for example, the cluster-aware-base-calling system 106 calculates various multi-cluster-phasing coefficients (g) corresponding to a previous cycle (g₋₁), a current cycle (g₀), and a subsequent cycle (g₁). As with the cluster-specific-phasing coefficients, the multi-cluster-phasing coefficients (g) sum to unity and are non-negative as represented by the function Σᵢgᵢ(c)=1,gᵢ≥0. As illustrated in FIG. 6, the cluster-aware-base-calling system 106 adjusts the signal based on both the cluster-specific-phasing correction (including cluster-specific-phasing coefficient) and the multi-cluster-phasing correction (including the multi-cluster-phasing coefficient).

In some embodiments, the cluster-aware-base-calling system 106 applies both the cluster-specific coefficient operation 606 and the multi-cluster coefficient operation 608 to a cluster. Additionally, or alternatively, the cluster-aware-base-calling system 106 applies the multi-cluster coefficient operation 608 but not the cluster-specific coefficient operation 606 to some clusters. In particular, in some embodiments, the cluster-aware-base-calling system 106 adjusts signals from one or more clusters based on a multi-cluster-phasing correction without a cluster-specific-phasing correction. For example, as mentioned previously, signals for nucleotide bases preceding an error-inducing sequence may not require cluster-specific-phasing corrections as the signals have not been affected by the error-inducing sequence. Accordingly, in some embodiments, the cluster-aware-base-calling system 106 identifies, for an additional cluster of oligonucleotides, a different read position preceding the error-inducing sequence within a different nucleotide-fragment read. The cluster-aware-base-calling system 106 further detects an additional signal from labeled nucleotide bases within the additional cluster of oligonucleotides during a cycle corresponding to the different read position. The cluster-aware-base-calling system 106 then adjusts the additional signal based on a multi-cluster phasing correction without a cluster-specific-phasing correction for the additional cluster of oligonucleotides.

In yet other embodiments, the cluster-aware-base-calling system 106 applies the cluster-specific coefficient operation 606 to a signal for a given cluster without performing the multi-cluster coefficient operation 608. For example, in some cases, the cluster-aware-base-calling system 106 applies a cluster-specific-phasing coefficient and a cluster-specific-pre-phasing coefficient (or other parameters) for a given cluster to a signal for the given cluster without applying parameters resulting from multi-cluster coefficient operations. Accordingly, when processing clusters within a nucleotide-sample slide, the cluster-aware-base-calling system 106 can apply a cluster-specific-phasing correction (without a multi-cluster-phasing correction) to to a signal for a given cluster, but apply a cluster-specific-phasing correction and a multi-cluster-phasing correction to a signal for a different cluster.

As previously mentioned, the cluster-aware-base-calling system 106 adjusts the signal based on cluster-specific-phasing coefficients and multi-cluster-phasing coefficients as part of the signal processing 604. In particular, and as illustrated in FIG. 6, the cluster-aware-base-calling system 106 performs the multi-cluster-phasing correction 610 as part of the signal processing 604. The cluster-aware-base-calling system 106 utilizes multi-cluster phasing coefficients generated from the multi-cluster coefficient operation 608 together with an algorithm (such as an FIR algorithm) to perform the multi-cluster-phasing correction 610. For example, the cluster-aware-base-calling system 106 adjusts a signal based on corrections (γ) corresponding to a previous cycle (y₋₁), a current cycle (γ₀), and a subsequent cycle (γ₁).

As further illustrated in FIG. 6, the cluster-aware-base-calling system 106 performs cluster-specific-phasing correction and base calling 612 as part of the signal processing 604. In particular, as part of the cluster-specific-phasing correction and the base calling 612, the cluster-aware-base-calling system 106 utilizes the cluster-specific-phasing coefficients generated as part of the cluster-specific coefficient operation 606 to estimate and apply cluster-specific-phasing corrections to the signal. In some embodiments, the cluster-aware-base-calling system 106 utilizes the cluster-specific-phasing coefficients together with an algorithm, such as an FIR algorithm, to perform the cluster-specific phasing correction. Furthermore, and as illustrated in FIG. 6, the cluster-aware-base-calling system 106 also performs base calling. In particular, the cluster-aware-base-calling system 106 generates nucleotide base calls based on the adjusted signals.

As previously mentioned, the cluster-aware-base-calling system 106 can determine the cluster-specific-phasing coefficient and the cluster-specific-pre-phasing coefficient utilizing several models or algorithms. More specifically, the cluster-aware-base-calling system 106 can utilize various models to perform the cluster-specific coefficient operation 606. In particular, the cluster-aware-base-calling system 106 can utilize a Linear Equalizer (LE), Decision Feedback Equalizer (DFE), a Maximum Likelihood Sequence Estimator (MLSE), or a forward-backward model to determine a cluster-specific-phasing coefficient and a cluster-specific-pre-phasing coefficient. Furthermore, the cluster-aware-base-calling system 106 may utilize a machine learning model, such as a multilayer perceptron, to determine the coefficients.

FIGS. 7A-7C and the corresponding paragraphs detail how the cluster-aware-base-calling system 106 utilizes an LE, DFE, or MLSE in accordance with one or more embodiments. Generally, the cluster-aware-base-calling system 106 can use various receiver types and computing architectures to estimate cluster-specific-phasing coefficients and cluster-specific-pre-phasing coefficients. More specifically, the cluster-aware-base-calling system 106 can generate and update coefficients over time within the course of a sequencing run. As indicated above, the cluster-aware-base-calling system 106 can utilize at least one of the three following models or algorithms as a receiver: LE, DFE, and MLSE. In some embodiments, the cluster-aware-base-calling system 106 utilizes a forward-backward model and/or a machine learning model to estimate cluster-specific-phasing coefficients and cluster-specific-pre-phasing coefficients. Additionally, in some embodiments, the cluster-aware-base-calling system 106 derives cluster-specific-phasing coefficients and cluster-specific-pre-phasing coefficients using least square error or other optimization.

The cluster-aware-base-calling system 106 can further utilize a real-time (or near real-time) computing architecture or a buffered computing architecture. The cluster-aware-base-calling system 106 utilizes a real-time computing architecture to output final base calls in each cycle without access to all future cycle data. For example, in some embodiments, the cluster-aware-base-calling system 106 needs only limited signal data to utilize real-time computing architecture. Additionally, or alternatively, the cluster-aware-base-calling system 106 utilizes a buffered computing architecture. The cluster-aware-base-calling system 106 utilizes a buffered computing architecture by utilizing signal data from all cycles before making final base calls. For example, the cluster-aware-base-calling system 106 can utilize a buffered computing architecture to generate cluster-specific-phasing corrections for a cluster based on signal data from all previous and subsequent cycles. The cluster-aware-base-calling system 106 can combine different receiver types with different compute architectures. For instance, the cluster-aware-base-calling system 106 can utilize a simple real time linear equalizer or the most complex buffered MLSE.

Generally, real-time computing architectures limit computing complexity by only using real-time (or near-real time) information. To illustrate, when the cluster-aware-base-calling system 106 utilizes a real-time computing architecture, the cluster-aware-base-calling system 106 only requires signal data for one or more previous cycles, a current cycle, and one or more subsequent cycles. In some embodiments, the cluster-aware-base-calling system 106 utilizes a set of signaling data from the previous cycle and a set of signaling data from the subsequent data. Because the real-time computing architecture is more computationally efficient, the cluster-aware-base-calling system 106 can perform operations utilizing the real-time computing architecture utilizing a process of a sequencing machine or device, such as the sequencing device 114.

By contrast, in some embodiments, the cluster-aware-base-calling system 106 determines cluster-specific-phasing corrections offline after a sequencing device has determined nucleotide-fragment reads for clusters of oligonucleotides on a nucleotide-sample slide. For instance, in some cases using MLSE or a machine learning model, the cluster-aware-base-calling system 106 determines cluster-specific-phasing coefficients and cluster-specific-pre-phasing coefficients for a given cluster— and adjusts signals corresponding to the given cluster—on a different computing device after a sequencing device has determined nucleotide-fragment reads for the given cluster.

In contrast, buffered computing architecture tends to require more computing resources. However, the cluster-aware-base-calling system 106 may generate more accurate results by utilizing a buffered computing architecture. To illustrate, by utilizing a buffered computing architecture, the cluster-aware-base-calling system 106 processes a large number of clusters and cycles in parallel. This type of processing requires a great amount of storage, communication, and computing resources for per-cluster phasing and pre-phasing estimations. However, utilizing buffered computing architecture may also yield more accurate results as the cluster-aware-base-calling system 106 processes signaling data for all cycles. In some embodiments, the cluster-aware-base-calling system 106 performs buffered computing when the sequencing machine or device is online and actively communicating with a central processing system.

As mentioned, FIG. 7A illustrates the cluster-aware-base-calling system 106 utilizing a Linear Equalizer (LE) to determine the cluster-specific-phasing coefficient and the cluster-specific-pre-phasing coefficient. Generally, LE is a linear filter that can be designed or optimized to suppress intersymbol interference (ISI) or to filter out noise. ISI refers to a form of distortion of a signal in which one symbol interferes with subsequent symbols. The effects of other symbols can have similar effects as noise, thus making communication less reliable. The cluster-aware-base-calling system 106 can optimize the LE to find an appropriate tradeoff between suppressing ISI and minimizing noise amplification. In some embodiments, the cluster-aware-base-calling system 106 utilizes a linear equalizer implemented as an FIR filter. Utilizing such an equalizer, the cluster-aware-base-calling system 106 linearly weights current and previous values of input signals by a filter coefficient. For example, in some embodiments, the current and previous values comprise current and previous signals from a cluster. The cluster-aware-base-calling system 106 further sums the weighted current and previous values to generate an adjusted signal.

FIG. 7A illustrates a linear equalizer architecture 700 in accordance with one or more embodiments. Generally, the cluster-aware-base-calling system 106 enters input signal x into the linear equalizer architecture 700 to generate an adjusted signal z. As previously described, h represents cluster-specific-phasing coefficients. Accordingly, h(D) represents a first filter. Additive noise is represented by n˜CN(0, σ²). As further illustrated in FIG. 7A, w represents a weight, and w(D) represents a second filter. The cluster-aware-base-calling system 106 further utilizes a decision device 702 to process the signal to generate an adjusted signal z.

To determine h in the LE structure shown in FIG. 7A, let S(f) be the frequency-domain SNR:

$$S ( f ) = \frac{{\vert F ( h ) \vert}^{2}}{{σ}^{2}}$$

where F(h) represents the Fourier transform of h(D). The cluster-aware-base-calling system 106 can generate a measure of signal quality by determining the Signal to Interference plus Noise Ratio (SINR). Assuming Gaussian noise, the SINR ratio can be used to derive error rate for a binary signal or other modulation type. For an ideal infinite-length unbiased minimum-mean-squared-error linear equalizer (U-MMSE-LE), it can be shown that

SINR_{U-MMSE-LE}=(∫_{-0.5}^{0.5}(1+S(f))⁻¹)−1.

The error rate can be closely approximated by the following:

$${P}_{error} ≃ 2 Q ( \sqrt{{SINR}_{U - MMSE - LE}} ) , where$$

$$Q ( x ) = \frac{1}{\sqrt{2 π}} {\int }_{x}^{∞} {e}^{- {x}^{\frac{2}{2}}} dx .$$

where Pₑᵣᵣₒᵣ represents the transmit power of the error. As suggested by FIG. 7A and the corresponding functions, given the signal and noise levels across the frequency band, the cluster-aware-base-calling system 106 calculates the total SNR after receiver processing and subsequently translates the SNR into an error rate estimation.

In some embodiments, the cluster-aware-base-calling system 106 utilizes a 3-tap LE to generate a previous-cycle weight, a subsequent-cycle weight, and a current-cycle weight. In particular, the cluster-aware-base-calling system 106 generates a previous-cycle weight estimating a phasing effect of the nucleotide base for the previous cycle based on the cluster-specific-phasing coefficient. The cluster-aware-base-calling system 106 also generates a subsequent-cycle weight estimating a pre-phasing effect of the nucleotide base for the subsequent cycle based on the cluster-specific-pre-phasing coefficient. Further, the cluster-aware-base-calling system 106 also generates a current-cycle weight estimating the phasing effect and the pre-phasing effect based on the cluster-specific-phasing coefficient and the cluster-specific-pre-phasing coefficient.

In some embodiments, the cluster-aware-base-calling system 106 determines a previous-cycle weight (w₋₁), a current cycle weight (w₀), and a subsequent-cycle weight (w₁). Generally, the cluster-aware-base-calling system 106 can optimize parameters using an optimization algorithm, such as least squares error or another optimization algorithm. For example, the cluster-aware-base-calling system 106 can generate decision directed minimum least squares estimates.

After generating decision directed minimum least squares estimates or otherwise optimizing parameters, the cluster-aware-base-calling system 106 may then calculate a cluster-specific-phasing coefficient (a) and a cluster-specific-pre-phasing coefficient (b) using intermediate statistics. In particular, the cluster-aware-base-calling system 106 utilizes intermediate statistics that are part of minimizing the squared error across several cycles and across one or more channels. Instead of maintaining all values per cycle per channel, the cluster-aware-base-calling system 106 efficiently accumulates the running statistics.

Based on the cluster-specific-phasing coefficient (a) and the cluster-specific-pre-phasing coefficient (b), the cluster-aware-base-calling system 106 then determines the previous-cycle weight (w₋₁), the current cycle weight (w₀), and the subsequent-cycle weight (w₁). The cluster-aware-base-calling system 106 applies each of the estimated weights to the signals from each cluster. In some embodiments, the cluster-aware-base-calling system 106 estimates the weights (w) as follows:

{w₋₁,w₀,w₁},={−a,1+a+b,−b}

As the function above and other functions herein suggest, in some embodiments, the cluster-aware-base-calling system 106 can determine a cluster-specific-phasing coefficient and a cluster-specific-pre-phasing coefficient (and corresponding weights) for a given cluster of oligonucleotides at on sequencing cycle and then determine an updated cluster-specific-phasing coefficient and an updated cluster-specific-pre-phasing coefficient (and corresponding weights) for the given cluster of oligonucleotides at a subsequent sequencing cycle, and so on and so forth for each subsequent cycle. Indeed, the cluster-aware-base-calling system 106 can re-determine and change cluster-specific-phasing coefficients and cluster-specific-pre-phasing coefficients for a given cluster of oligonucleotides over the course of determining nucleotide-base calls for a nucleotide-fragment read corresponding to the given cluster. Accordingly, in some cases, the cluster-aware-base-calling system 106 does not simply determine a cluster-specific-phasing coefficient and a cluster-specific-pre-phasing coefficient once for a given cluster, but repeatedly determines and updates such a cluster-specific-phasing coefficient and a cluster-specific-pre-phasing coefficient for a given cluster as sequencing cycles progress.

As previously described, the cluster-aware-base-calling system 106 can also utilize a Decision Feedback Equalizer (DFE) to determine the cluster-specific-phasing coefficient and the cluster-specific-pre-phasing coefficient. FIG. 7B and the corresponding paragraphs illustrate how the cluster-aware-base-calling system 106 utilizes DFE and a decision feedback equalizer architecture 706 in accordance with one or more embodiments. Generally, DFE is a form of non-linear equalization that relies on decisions about the levels of previous signals to correct the current signal. In particular, the cluster-aware-base-calling system 106 utilizes a DFE to employ previous decisions as training sequences. This allows the cluster-aware-base-calling system 106 to account for distortion in the current signal that is caused by the previous signals. In some embodiments, the DFE comprises a feed forward filter (FFF) and a feedback filter (FBF). The FFF can comprise a linear equalizer whose output is given to a decision device. The FBF is driven by the output of the decision device.

In particular, and as illustrated in FIG. 7B, the cluster-aware-base-calling system 106 enters the input signal x into the decision feedback equalizer architecture 706 to generate an adjusted signal \hat{x}. As illustrated, the decision feedback equalizer architecture 706 includes a feed forward filter h(D) corresponding to the cluster-specific-phasing coefficients h. Additive noise for the signal x is represented by n˜CN(0, σ²). The decision feedback equalizer architecture 706 further includes a decision device 708 that processes the signal. Generally, the decision device 708 determines whether the noise exceeds a pre-determined value or not. The decision feedback equalizer architecture 706 further includes feedback filter b(D).

For an infinite-length unbiased minimum-mean-squared-error decision feedback equalizer (U-MMSE-DFE), it can be shown that

$${SINR}_{U - MMSE - DFE} = exp ( {\int }_{- 0.5}^{0.5} log ( 1 + S ( f ) ) df ) - 1$$

assuming correct (genie-aided) decisions. S(f) represents the ratio of (i) the squared magnitude of the Fourier transform of the channel over (ii) noise power across the frequency band. Given s(f), the cluster-aware-base-calling system 106 can calculate the SINR at or using a slicer, which the cluster-aware-base-calling system 106 utilizes to estimate the bit error rate for the binary signal. As mentioned previously, the cluster-aware-base-calling system 106 can generate a measure of signal quality by determining the Signal to Interference plus Noise Ratio (SINR). One can see that this expression is related to the Shannon Limit

C=∫_{-0.5}^{0.5} log(1+S(f))df=log(1+SINR_{U-MMSE-DFE})

The channel capacity (C) represents the theoretical tightest upper bound on the information rate of data that can be communicated at an arbitrarily low error rate using an average received signal power (S) through an analog communication channel subject to additive white Gaussian noise. In a real-world communication system, the Shannon Limit can be approached by combining strong codes, Gaussian constellation shaping, and precoding. For uncoded QPSK, error propagation is unavoidable and the error rate is lower bounded by:

Pₑᵣᵣₒᵣ>˜2Q(\sqrt{SINR_{U-MMSE-DFE}})

where Pₑᵣᵣₒᵣ represents the transmit power of the error.

In yet other embodiments, the cluster-aware-base-calling system 106 utilizes a third type of receiver, a Maximum Likelihood Sequence Estimator (MLSE), to determine the cluster-specific-phasing coefficient and the cluster-specific-pre-phasing coefficient. FIG. 7C illustrates a maximum likelihood sequence estimator architecture 710 in accordance with one or more embodiments. MLSE is a nonlinear estimation technique that replaces an equalizing filter with an MLSE estimation. Generally, the cluster-aware-base-calling system 106 utilizes the MLSE to test all possible data sequences (rather than decoding each received signal by itself), and chooses the output signal with the maximum probability as the output. MLSE uses a Viterbi decoder 712 to determine the probabilities of all possible transmitted sequences. As illustrated in FIG. 7C, the cluster-aware-base-calling system 106 inputs the input signal x into the maximum likelihood sequence estimator architecture 710 to generate an adjusted signal \hat{x}. The maximum likelihood sequence estimator architecture 710 includes a filter h(D) corresponding to the cluster-specific-phasing coefficients h. Additive noise for the signal x is represented by n˜CN(0, σ²).

As illustrated in FIG. 7C, the error rate is bounded by the Matched Filter Bound (MFB) as follows:

$${P}_{error} \geq 2 Q ( \sqrt{{SNR}_{MFB}} )$$

$${SNR}_{MFB} = \frac{{\sum }_{i} {\vert {h}_{i} \vert}^{2}}{{σ}^{2}} = {\int }_{- 0.5}^{0.5} S ( f ) df$$

Where SNR represents a Signal to Noise Ratio and Pₑᵣᵣₒᵣ represents the transmit power of the error. Generally, the SNR compares the level of a desired signal to the level of background noise. As indicated by FIG. 7C and the corresponding functions, the cluster-aware-base-calling system 106 utilizes Parseval's theorem to determine a total signal power by summing the response in the time domain. The total signal power can be identical or equal to total power in the frequency domain. Once the cluster-aware-base-calling system 106 determines SNR, the cluster-aware-base-calling system 106 calculates error bounds. In the functions above corresponding to FIG. 7C, the number of states is given by N^{length(h)-1}, where N is the number of constellation points. For a square constellation with uncorrelated noise, the two SBS channels can be processed independently, reducing the number of states.

As indicated above, the cluster-aware-base-calling system 106 can utilize other models in addition to the receivers LE, DFE, and MLSE illustrated in FIGS. 7A-7C. More specifically, the cluster-aware-base-calling system 106 can utilize other Hidden Markov Models (HMMs) in addition to those listed above. For example, in some embodiments, the cluster-aware-base-calling system 106 can utilize a forward-backward model to generate a maximum a posteriori probability (MAP) estimate. A forward-backward model computes an a posteriori maximum path probability for each state at a given time. Generally, the forward-backward model makes use of dynamic programming principles to compute values required to obtain the posterior marginal distribution in two passes. The first pass goes forward in time while the second pass goes backward in time.

In addition to the models listed above, the cluster-aware-base-calling system 106 can determine a cluster-specific-phasing coefficient and a cluster-specific-pre-phasing coefficient utilizing a machine learning model. Generally, the cluster-aware-base-calling system 106 can use a machine learning model to estimate cluster-specific-phasing coefficients and cluster-specific-pre-phasing coefficients, adjust resulting signals, or directly adjust nucleotide-base calls. To illustrate, in some embodiments, the cluster-aware-base-calling system 106 utilizes a sequence-to-sequence machine learning model based on convolutional layers. Additionally, or alternatively, the cluster-aware-base-calling system 106 may utilize a Recurrent Neural Network (RNN), such as a Long Short-Term Memory (LSTM), to estimate cluster-specific-phasing coefficients and cluster-specific-pre-phasing coefficients. In yet other embodiments, the cluster-aware-base-calling system 106 utilizes an attention-based model.

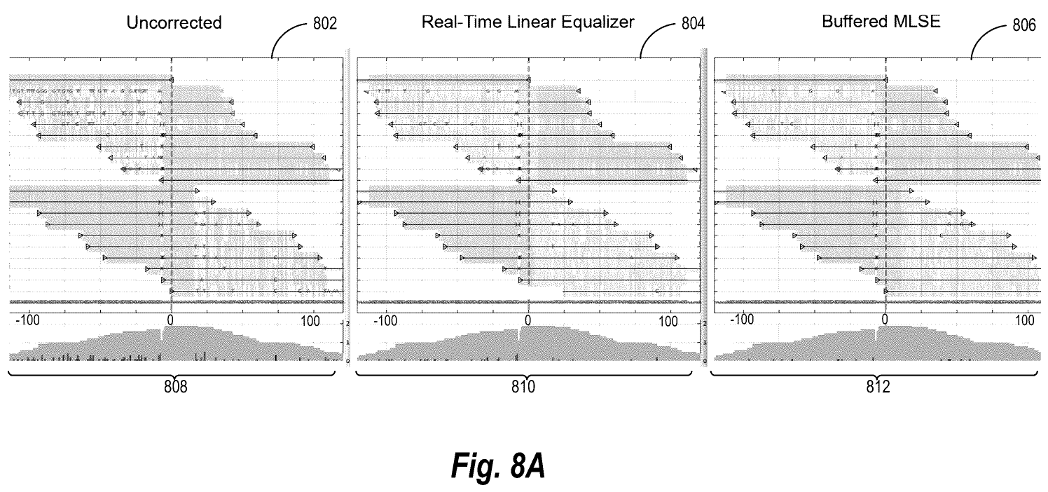

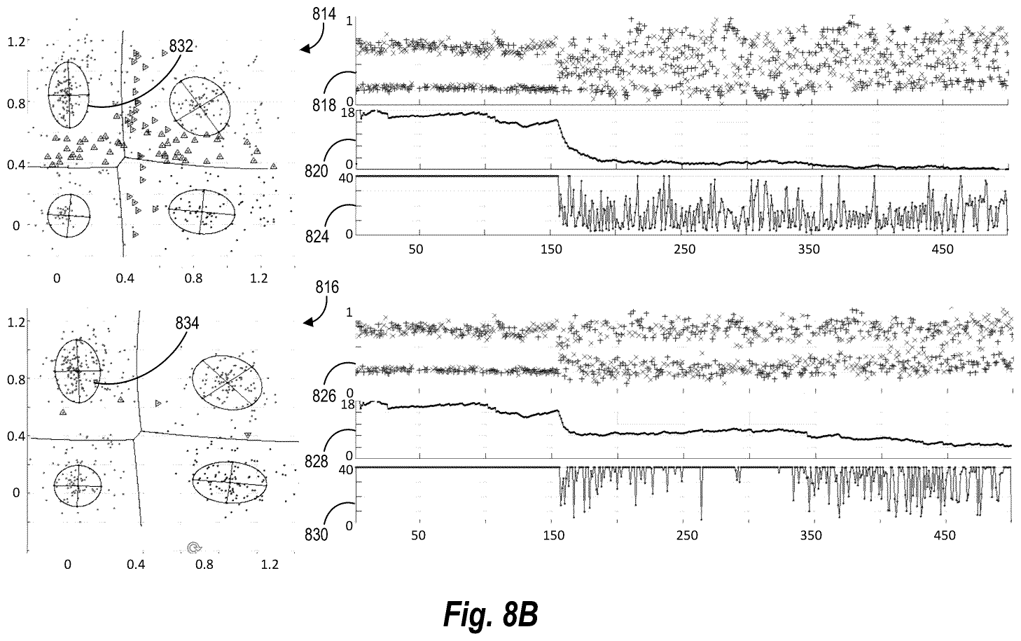

FIGS. 7A-7C illustrate different receivers that the cluster-aware-base-calling system 106 utilizes to determine the cluster-specific-phasing correction in accordance with one or more embodiments. FIGS. 8A-8B illustrate technical improvements resulting from the cluster-aware-base-calling system 106 utilizing a real-time LE and a buffered MLSE in accordance with one or more embodiments. In particular, FIG. 8A illustrates example read pileups corresponding with no correction, real-time LE, and buffered MLSE. FIG. 8B illustrates a cluster demonstrating large gains in secondary sequencing metrics from cluster-specific-phasing corrections.

As mentioned, FIG. 8A illustrates three read pileups corresponding to no correction, a real-time LE, and a buffered MLSE. In particular, FIG. 8A illustrates an uncorrected read pileup 802, a read pileup 804 with nucleotide-base calls from signals adjusted using a cluster-specific-phasing correction by a real-time linear equalizer, and a read pileup 806 with nucleotide-base calls from signals adjusted using a cluster-specific-phasing correction by a buffered MLSE. The uncorrected read pileup 802 is similar to the read pileup 200 illustrated in FIG. 2A. In particular, the uncorrected read pileup 802 reflects that base-call accuracy degrades after an error-inducing sequence. To illustrate, in FIG. 8A, an uncorrected error type counter 808 indicates an increased occurrence of base-call errors surrounding the error-inducing sequence.

FIG. 8A further illustrates that by using a real-time linear equalizer, the cluster-aware-base-calling system 106 decreases the occurrence of base-call errors. In particular, the read pileup 804 with nucleotide-base calls from signals adjusted using a cluster-specific-phasing correction by a real-time linear equalizer indicates fewer base-call errors, even surrounding an error-inducing sequence, than the uncorrected read pileup 802. For example, when compared with the uncorrected error type counter 808, a linear equalizer error type counter 810 includes both fewer and shorter bars. As illustrated in FIG. 8A, by using real-time LE to determine cluster-specific-phasing corrections, the cluster-aware-base-calling system 106 accurately determines around 70% of the nucleotide-base calls that are shown as errors (or incorrect nucleotide-base calls) in the uncorrected read pileup 802. However, some base-call errors highly correlated with the error-inducing sequence are still present. For example, the read pileup 804 still includes several base-call errors in the bases immediately surrounding the error-inducing sequence.

As previously mentioned, while it is often less computationally efficient, the cluster-aware-base-calling system 106 can improve the accuracy of nucleotide-base calls by using a buffered MLSE, even relative to using the real-time linear equalizer. FIG. 8A further illustrates the read pileup 806 with a buffered MLSE error type counter 812. The buffered MLSE error type counter 812 indicates that, by using buffered MLSE to determine cluster-specific-phasing corrections, the cluster-aware-base-calling system 106 accurately determines around 85% of the nucleotide-base calls that are shown as errors (or incorrect nucleotide-base calls) in the uncorrected read pileup 802.

While FIG. 8A illustrates improvements in nucleotide-base call accuracy based on adjusting signals according to a cluster-specific-phasing correction, FIG. 8B illustrates improvements in secondary sequencing metrics based on adjusting signals according to a cluster-specific-phasing correction in accordance with one or more embodiments. In particular, FIG. 8B illustrates a comparison of various secondary sequencing metrics resulting from uncorrected signals and signals corrected by a cluster-specific-phasing correction utilizing LE. For example, FIG. 8B illustrates secondary sequencing metrics corresponding to an uncorrected intensity. In particular, FIG. 8B includes an uncorrected graph 814, an uncorrected intensity spread 818, an uncorrected SNR graph 820, and an uncorrected quality score graph 824. FIG. 8B also illustrates secondary sequencing metrics from signals adjusted by a cluster-specific-phasing correction utilizing LE. In particular, FIG. 8B includes an adjusted graph 816, an adjusted intensity spread 826, an adjusted SNR graph 828, and an adjusted quality score graph 830.

As illustrated in FIG. 8B, the utilization of LE enables the cluster-aware-base-calling system 106 to generate signals for nucleotide-base calls with better chastity for intensity-value boundaries than previous sequencing systems. In particular, FIG. 8B includes the uncorrected graph 814 including an uncorrected intensity-value boundary 832 and the adjusted graph 816 including an adjusted intensity-value boundary 834. As described previously, intensity-value boundaries correspond to each possible nucleotide base (e.g., A, T, C, or G). As illustrated in FIG. 8B, the cluster-aware-base-calling system 106 generates signals for nucleotide-base calls with better chastity values with respect to intensity-value boundaries in the adjusted graph 816 than in the uncorrected graph 814. As illustrated in FIG. 8B, the adjusted graph 816 shows fewer adjusted signals with values that do not pass the chastity filter. In particular, as a result of adjusting signals to account for phasing and pre-phasing, the cluster-aware-base-calling system 106 reduces the number of signals with values that fail the chastity filter. In contrast, the uncorrected graph 814 indicate a higher occurrence of noise or signals with values that fail the chastity filter as the triangles located outside of the uncorrected intensity-value boundary 832 outnumber the triangles outside of the adjusted intensity-value boundary 834 in the adjusted graph 816.

The uncorrected intensity spread 818 and the adjusted intensity spread 826 in FIG. 8B illustrate how the cluster-aware-base-calling system 106 clarifies signal intensity by adjusting signals based on cluster-specific phasing corrections. Generally, intensity spreads translate two channels of intensity to superimpose them on one axis. Ideally, the signals from the two channels should have good separation, which indicates a clarity of signals. As illustrated in FIG. 8B, the uncorrected intensity spread 818 indicates that signal intensity after an error-inducing sequence is jumbled. In contrast, the adjusted intensity spread 826 shows a clearer delineation of signals even following an error-inducing sequence.

As further illustrated in FIG. 8B, the cluster-aware-base-calling system 106 also improves SNR metrics by utilizing LE to determine cluster-specific-phasing corrections for adjusting signals. In particular, the uncorrected SNR graph 820 indicates a dramatic drop in SNR metric following an error-inducing sequence just after the read position 150. In contrast, the adjusted SNR graph 828 indicates a smaller decrease in SNR metric, even following an error-inducing sequence just after the read position 150. Thus, by utilizing LE, the cluster-aware-base-calling system 106 can improve SNR metrics.

FIG. 8B also illustrates an improvement in quality scores in cycles following an error-inducing sequence based on utilizing LE to determine cluster-specific-phasing corrections for adjusting signals. As illustrated, the uncorrected quality score graph 824 includes a dramatic drop in quality score. In some embodiments, the cluster-aware-base-calling system 106 measures a Phred (Q30) quality score. In contrast to the uncorrected quality score graph 824 that shows occasional quality score peaks in cycles following an error-inducing sequence, the adjusted quality score graph 830 indicates consistently higher quality scores with occasional dips in the cycles following the error-inducing sequence.

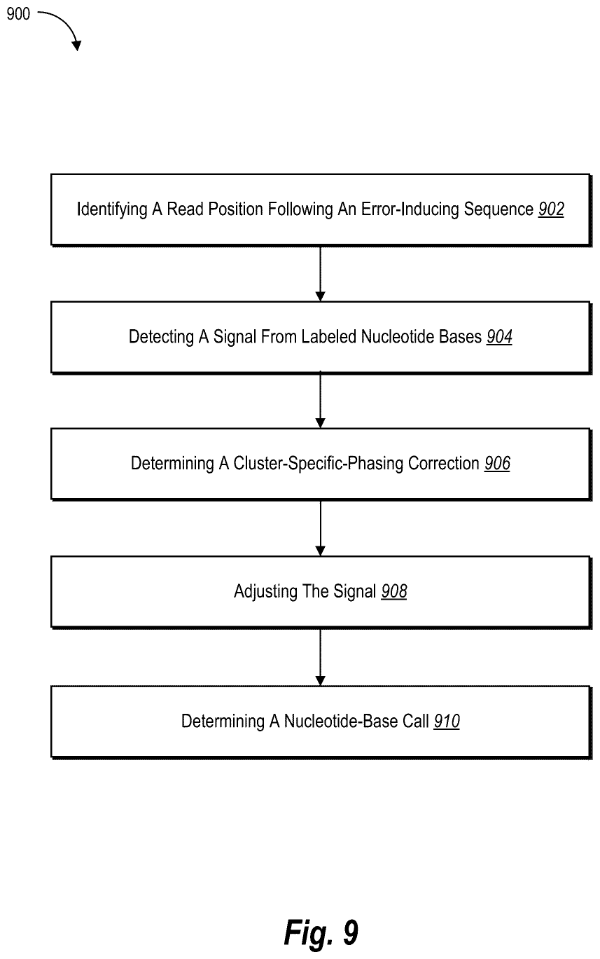

FIGS. 1-8B, the corresponding text, and the examples provide a number of different methods, systems, devices, and non-transitory computer readable media of the cluster-aware-base-calling system 106. In addition to the foregoing, one or more embodiments can also be described in terms of flowcharts comprising acts for accomplishing a particular result, such as the flowchart of acts as shown in FIG. 9. Additionally, the acts described herein may be repeated or performed in parallel with one another or in parallel with different instances of the same or similar acts.

FIG. 9 illustrates a flowchart of a series of acts 900 for determining a nucleotide-base call based on a cluster-specific-phasing correction. While FIG. 9 illustrates acts according to one embodiment, alternative embodiments may omit, add to, reorder and/or modify any of the acts shown in FIG. 9. The acts of FIG. 9 can be performed as part of a method. Alternatively, a non-transitory computer-readable medium can comprise instructions that, when executed by one or more processors, cause a computing device to perform the acts of FIG. 9. In some embodiments, a system can perform the acts of FIG. 9.

In one or more embodiments, the series of acts 900 is implemented on one or more computing devices, such as the computing device illustrated in FIG. 10. In addition, in some embodiments, the series of acts 900 is implemented in a digital environment for sequencing nucleic-acid polymers. As illustrated in FIG. 9, the series of acts 900 includes an act 902 of identifying a read position following an error-inducing sequence, an act 904 of detecting a signal from labeled nucleotide bases, an act 906 of determining a cluster-specific-phasing correction, an act 908 of adjusting the signal, and an act 910 of determining a nucleotide-base call.

The series of acts 900 illustrated in FIG. 9 includes the act 902 of identifying a read position following an error-inducing sequence. In particular, the act 902 comprises identifying, for a cluster of oligonucleotides, a read position following an error-inducing sequence within one or more nucleotide-fragment reads. In one or more embodiments, the error-inducing sequence comprises a sequence of one or more repeated nucleotide bases or a sequence motif. Furthermore, in some embodiments, the sequence of one or more repeated nucleotide bases or the sequence motif comprise a homopolymer of a same nucleotide base, a near-homopolymer, a guanine quadruplex, a variable number tandem repeat (VNTR), a dinucleotide-repeat sequence, a trinucleotide-repeat sequence, an inverted-repeat sequence, a minisatellite sequence, a microsatellite sequence, or a palindromic sequence. In one or more embodiments, the error-inducing sequence comprises a sequence of one or more repeated nucleotide bases or a direction-specific sequence motif.

FIG. 9 further illustrates the act 904 of detecting a signal from labeled nucleotide bases. In particular, the act 904 comprises detecting a signal from labeled nucleotide bases within the cluster of oligonucleotides during a cycle corresponding to the read position.

The series of acts 900 illustrated in FIG. 9 further comprises the act 906 of determining a cluster-specific-phasing correction. In particular, the act 906 comprises determining, for the cluster of oligonucleotides, a cluster-specific-phasing correction to correct the signal for estimated phasing and estimated pre-phasing. In some embodiments, the act 906 comprises determining, for the cluster of oligonucleotides, a cluster-specific-phasing coefficient corresponding to a nucleotide base for a previous cycle and a cluster-specific-pre-phasing coefficient corresponding to a nucleotide base for a subsequent cycle. In some embodiments, the act 906 comprises determining, for the cluster of oligonucleotides, a cluster-specific-phasing correction to correct the signal for phasing and pre-phasing. In one or more embodiments, determining the cluster-specific-phasing correction comprises: determining, for the cluster of oligonucleotides, a cluster-specific-phasing coefficient corresponding to a nucleotide base for a previous cycle immediately preceding the cycle and a cluster-specific-pre-phasing coefficient corresponding to a nucleotide base for a subsequent cycle immediately following the cycle; and determining the cluster-specific-phasing correction based on the cluster-specific-phasing coefficient and the cluster-specific-pre-phasing coefficient.

In some embodiments, the act 906 further comprises determining the cluster-specific-phasing correction by: determining, for the cluster of oligonucleotides, a cluster-specific-phasing coefficient corresponding to a nucleotide base for a previous cycle and a cluster-specific-pre-phasing coefficient corresponding to a nucleotide base for a subsequent cycle; and determining the cluster-specific-phasing correction based on the cluster-specific-phasing coefficient and the cluster-specific-pre-phasing coefficient. Furthermore, in some embodiments, the act 906 further comprises determining the cluster-specific-phasing correction based on the cluster-specific-phasing coefficient and the cluster-specific-pre-phasing coefficient by: generating a previous-cycle weight estimating a phasing effect of the nucleotide base for the previous cycle based on the cluster-specific-phasing coefficient; generating a subsequent-cycle weight estimating a pre-phasing effect of the nucleotide base for the subsequent cycle based on the cluster-specific-pre-phasing coefficient; generating a current-cycle weight estimating the phasing effect and the pre-phasing effect for the cycle based on the cluster-specific-phasing coefficient and the cluster-specific-pre-phasing coefficient; and determining the cluster-specific-phasing correction based on the previous-cycle weight, the subsequent-cycle weight, and the current-cycle weight. In some cases, determining the cluster-specific-phasing correction is further based on a signal intensity corresponding to the previous cycle, a signal intensity corresponding to the current cycle, and a signal intensity corresponding to the subsequent cycle.

Similarly, in some embodiments, the act 906 further comprises adjusting the signal based on the cluster-specific-phasing coefficient and the cluster-specific-pre-phasing coefficient by: generating a previous-cycle weight estimating a phasing effect of the nucleotide base for the previous cycle based on the cluster-specific-phasing coefficient; generating a subsequent-cycle weight estimating a pre-phasing effect of the nucleotide base for the subsequent cycle based on the cluster-specific-pre-phasing coefficient; generating a current-cycle weight estimating the phasing effect and the pre-phasing effect for the cycle based on the cluster-specific-phasing coefficient and the cluster-specific-pre-phasing coefficient; determining a cluster-specific-phasing correction based on the previous-cycle weight, the subsequent-cycle weight, and the current-cycle weight; and applying the cluster-specific-phasing correction to the signal.

Furthermore, in some embodiments, the act 906 further comprises determining the cluster-specific-phasing correction by: determining, for the cluster of oligonucleotides, a set of cluster-specific-phasing coefficients corresponding to a set of nucleotide bases for a set of previous cycles; determining, for the cluster of oligonucleotides, a set of cluster-specific-pre-phasing coefficients corresponding to a set of nucleotide bases for a set of subsequent cycles; and determining the cluster-specific-phasing correction based on the set of cluster-specific-phasing coefficients and the set of cluster-specific-pre-phasing coefficients. In some embodiments the act 906 further comprises determining the cluster-specific-phasing correction utilizing a processor of a sequencing device.

In some embodiments, the act 906 further comprises determining, on a sequencing machine of the system, the cluster-specific-phasing coefficient and the cluster-specific-pre-phasing coefficient utilizing a Linear Equalizer, Decision Feedback Equalizer, Maximum Likelihood Sequence Estimator, forward-backward model, or machine learning model. Additionally, in some embodiments, the act 906 further comprises determining the cluster-specific-phasing coefficient and the cluster-specific-pre-phasing coefficient after a sequencing run.

Additionally, in one or more embodiments, the act 906 further comprises determining, for the cluster of oligonucleotides, a set of cluster-specific-phasing coefficients corresponding to a set of nucleotide bases for a set of previous cycles immediately preceding the cycle; determining, for the cluster of oligonucleotides, a set of cluster-specific-pre-phasing coefficients corresponding to a set of nucleotide bases for a set of subsequent cycles immediately following the cycle; and determining the cluster-specific-phasing correction based on the set of cluster-specific-phasing coefficients and the set of cluster-specific-pre-phasing coefficients.

As illustrated in FIG. 9, the series of acts 900 includes the act 908 of adjusting the signal. In particular, the act 908 comprises adjusting the signal based on the cluster-specific-phasing correction. In some embodiments, the act 908 comprises adjusting the signal based on the cluster-specific-phasing coefficient and the cluster-specific-pre-phasing coefficient. Additionally, in some embodiments, the act 908 further comprises adjusting the signal by: determining, for the cluster of oligonucleotides, an additional cluster-specific-phasing coefficient corresponding to an additional nucleotide base for an additional previous cycle; determining, for the cluster of oligonucleotides, an additional cluster-specific-pre-phasing coefficient corresponding to an additional nucleotide base for an additional subsequent cycle; and determining a cluster-specific-phasing correction based on the cluster-specific-phasing coefficient, the additional cluster-specific-phasing coefficient, the cluster-specific-pre-phasing coefficient, and the additional cluster-specific-pre-phasing coefficient.

The series of acts 900 also includes the act 910 of determining a nucleotide-base call. In particular, the act 910 comprises determining a nucleotide-base call for the read position corresponding to the cluster of oligonucleotides based on the adjusted signal.

In one or more embodiments, the series of acts 900 includes additional acts of determining, for a set of clusters of oligonucleotides, a multi-cluster-phasing correction to correct signals from the set of clusters for estimated phasing and estimated pre-phasing; and adjusting the signal based on the cluster-specific-phasing correction or the multi-cluster-phasing correction. In some embodiments, the series of acts 900 includes the additional acts of determining, for a set of clusters of oligonucleotides, one or more of a multi-cluster-phasing coefficient for estimated phasing or a multi-cluster-pre-phasing coefficient for estimated pre-phasing; and adjusting the signal based on one or more of the multi-cluster-phasing coefficient, the cluster-specific-phasing coefficient, the multi-cluster-pre-phasing coefficient, or the cluster-specific-pre-phasing coefficient. In some embodiments, the series of acts 900 further includes the acts determining, for a set of clusters of oligonucleotides, a multi-cluster-phasing correction to correct signals from the set of clusters for phasing and pre-phasing; and adjusting the signal based on both the cluster-specific-phasing correction and the multi-cluster-phasing correction.

In one or more embodiments, the series of acts 900 includes an additional act of determining, for the cluster of oligonucleotides and a subsequent read position, a different cluster-specific-phasing correction to correct a signal for a subsequent cycle from the cluster of oligonucleotides for phasing and pre-phasing of the signal for the subsequent cycle.

In some embodiments, the series of acts 900 illustrated in FIG. 9 include additional acts of identifying, for an additional cluster of oligonucleotides, a different read position preceding the error-inducing sequence within a different nucleotide-fragment read; detecting an additional signal from labeled nucleotide bases within the additional cluster of oligonucleotides during a cycle corresponding to the different read position; and adjusting the additional signal based on a multi-cluster-phasing correction without a cluster-specific-phasing correction for the additional cluster of oligonucleotides.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

The SBS techniques described below can utilize single-read sequencing or paired-end sequencing. In single-rea sequencing, the sequencing device reads a fragment from one end to another to generate the sequence of base pairs. In contrast, during paired-end sequencing, the sequencing device begins at one read, finishes reading a specified read length in the same direction, and begins another read from the opposite end of the fragment.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeg™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the cluster-aware-base-calling system 106 can include software, hardware, or both. For example, the components of the cluster-aware-base-calling system 106 can include one or more instructions stored on a non-transitory computer readable storage medium and executable by processors of one or more computing devices (e.g., the user client device 108). When executed by the one or more processors, the computer-executable instructions of the cluster-aware-base-calling system 106 can cause the computing devices to perform the failure source identification methods described herein. Alternatively, the components of the cluster-aware-base-calling system 106 can comprise hardware, such as special purpose processing devices to perform a certain function or group of functions. Additionally, or alternatively, the components of the cluster-aware-base-calling system 106 can include a combination of computer-executable instructions and hardware.

Furthermore, the components of the cluster-aware-base-calling system 106 performing the functions described herein with respect to the cluster-aware-base-calling system 106 may, for example, be implemented as part of a stand-alone application, as a module of an application, as a plug-in for applications, as a library function or functions that may be called by other applications, and/or as a cloud-computing model. Thus, components of the cluster-aware-base-calling system 106 may be implemented as part of a stand-alone application on a personal computing device or a mobile device. Additionally, or alternatively, the components of the cluster-aware-base-calling system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer readable medium and executable by one or more computing devices (e.g., any of the media content access devices described herein). In general, a processor (e.g., a microprocessor) receives instructions, from a non-transitory computer readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 10 illustrates a block diagram of a computing device 1000 that may be configured to perform one or more of the processes described above. One will appreciate that one or more computing devices such as the computing device 1000 may implement the cluster-aware-base-calling system 106 and the sequencing system 104. As shown by FIG. 10, the computing device 1000 can comprise a processor 1002, a memory 1004, a storage device 1006, an I/O interface 1008, and a communication interface 1010, which may be communicatively coupled by way of a communication infrastructure 1012. In certain embodiments, the computing device 1000 can include fewer or more components than those shown in FIG. 10. The following paragraphs describe components of the computing device 1000 shown in FIG. 10 in additional detail.

In one or more embodiments, the processor 1002 includes hardware for executing instructions, such as those making up a computer program. As an example, and not by way of limitation, to execute instructions for dynamically modifying workflows, the processor 1002 may retrieve (or fetch) the instructions from an internal register, an internal cache, the memory 1004, or the storage device 1006 and decode and execute them. The memory 1004 may be a volatile or non-volatile memory used for storing data, metadata, and programs for execution by the processor(s). The storage device 1006 includes storage, such as a hard disk, flash disk drive, or other digital storage device, for storing data or instructions for performing the methods described herein.

The I/O interface 1008 allows a user to provide input to, receive output from, and otherwise transfer data to and receive data from computing device 1000. The I/O interface 1008 may include a mouse, a keypad or a keyboard, a touch screen, a camera, an optical scanner, network interface, modem, other known I/O devices or a combination of such I/O interfaces. The I/O interface 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1008 is configured to provide graphical data to a display for presentation to a user. The graphical data may be representative of one or more graphical user interfaces and/or any other graphical content as may serve a particular implementation.

The communication interface 1010 can include hardware, software, or both. In any event, the communication interface 1010 can provide one or more interfaces for communication (such as, for example, packet-based communication) between the computing device 1000 and one or more other computing devices or networks. As an example, and not by way of limitation, the communication interface 1010 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1010 may facilitate communications with various types of wired or wireless networks. The communication interface 1010 may also facilitate communications using various communication protocols. The communication infrastructure 1012 may also include hardware, software, or both that couples components of the computing device 1000 to each other. For example, the communication interface 1010 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A non-transitory computer readable storage medium comprising instructions that, when executed by at least one processor, cause a computing device to:
identify, for a cluster of oligonucleotides, a read position following an error-inducing sequence within one or more nucleotide-fragment reads;
detect a signal from labeled nucleotide bases within the cluster of oligonucleotides during a cycle corresponding to the read position;
determine, for the cluster of oligonucleotides, a cluster-specific-phasing correction to correct the signal for estimated phasing and estimated pre-phasing;
adjust the signal based on the cluster-specific-phasing correction; and
determine a nucleotide-base call for the read position corresponding to the cluster of oligonucleotides based on the adjusted signal.

2. The non-transitory computer readable storage medium of claim 1, wherein the error-inducing sequence comprises a sequence of one or more repeated nucleotide bases, a sequence motif, or a trigger sequence identified by a sequence recognition model.

3. The non-transitory computer readable storage medium of claim 2, wherein the sequence of one or more repeated nucleotide bases or the sequence motif comprise a homopolymer of a same nucleotide base, a near-homopolymer, a guanine quadruplex, a variable number tandem repeat (VNTR), a dinucleotide-repeat sequence, a trinucleotide-repeat sequence, an inverted-repeat sequence, a minisatellite sequence, a microsatellite sequence, or a palindromic sequence.

4. The non-transitory computer readable storage medium of claim 1, further comprising instructions that, when executed by the at least one processor, cause the computing device to determine the cluster-specific-phasing correction by:
determining, for the cluster of oligonucleotides, a cluster-specific-phasing coefficient corresponding to a nucleotide base for a previous cycle and a cluster-specific-pre-phasing coefficient corresponding to a nucleotide base for a subsequent cycle; and
determining the cluster-specific-phasing correction based on the cluster-specific-phasing coefficient and the cluster-specific-pre-phasing coefficient.

5. The non-transitory computer readable storage medium of claim 4, further comprising instructions that, when executed by the at least one processor, cause the computing device to determine the cluster-specific-phasing correction based on the cluster-specific-phasing coefficient and the cluster-specific-pre-phasing coefficient by:
generating a previous-cycle weight estimating a phasing effect of the nucleotide base for the previous cycle based on the cluster-specific-phasing coefficient;
generating a subsequent-cycle weight estimating a pre-phasing effect of the nucleotide base for the subsequent cycle based on the cluster-specific-pre-phasing coefficient;
generating a current-cycle weight estimating the phasing effect and the pre-phasing effect for the cycle based on the cluster-specific-phasing coefficient and the cluster-specific-pre-phasing coefficient; and
determining the cluster-specific-phasing correction based on the previous-cycle weight, the subsequent-cycle weight, and the current-cycle weight.

6. The non-transitory computer readable storage medium of claim 5, further comprising instructions that, when executed by the at least one processor, cause the computing device to determine the cluster-specific-phasing correction further based on a signal intensity corresponding to the previous cycle, a signal intensity corresponding to the cycle, and a signal intensity corresponding to the subsequent cycle.

7. The non-transitory computer readable storage medium of claim 1, further comprising instructions that, when executed by the at least one processor, cause the computing device to determine the cluster-specific-phasing correction by:
determining, for the cluster of oligonucleotides, a set of cluster-specific-phasing coefficients corresponding to a set of nucleotide bases for a set of previous cycles;
determining, for the cluster of oligonucleotides, a set of cluster-specific-pre-phasing coefficients corresponding to a set of nucleotide bases for a set of subsequent cycles; and
determining the cluster-specific-phasing correction based on the set of cluster-specific-phasing coefficients and the set of cluster-specific-pre-phasing coefficients.

8. The non-transitory computer readable storage medium of claim 1, further comprising instructions that, when executed by the at least one processor, cause the computing device to:
determine, for a set of clusters of oligonucleotides, a multi-cluster-phasing correction to correct signals from the set of clusters for estimated phasing and estimated pre-phasing; and
adjust the signal based on the cluster-specific-phasing correction or the multi-cluster-phasing correction.

9. The non-transitory computer readable storage medium of claim 1, further comprising instructions that, when executed by the at least one processor, cause the computing device to determine, for the cluster of oligonucleotides and a subsequent read position, a different cluster-specific-phasing correction to correct a signal for a subsequent cycle from the cluster of oligonucleotides for phasing and pre-phasing of the signal for the subsequent cycle.

10. The non-transitory computer readable storage medium of claim 1, further comprising instructions that, when executed by the at least one processor, cause the computing device to:
identify, for an additional cluster of oligonucleotides, a different read position preceding the error-inducing sequence within a different nucleotide-fragment read;
detect an additional signal from labeled nucleotide bases within the additional cluster of oligonucleotides during a cycle corresponding to the different read position; and
adjust the additional signal based on a multi-cluster-phasing correction without a cluster-specific-phasing correction for the additional cluster of oligonucleotides.

11. The non-transitory computer readable storage medium of claim 1, further comprising instructions that, when executed by the at least one processor, cause the computing device to determine the cluster-specific-phasing correction utilizing a processor of a sequencing device.

12. A system comprising:
at least one processor; and
a non-transitory computer readable medium comprising instructions that, when executed by the at least one processor, cause the system to:
identify, for a cluster of oligonucleotides, a read position following an error-inducing sequence within one or more nucleotide-fragment reads;
detect a signal from labeled nucleotide bases within the cluster of oligonucleotides during a cycle corresponding to the read position;
determine, for the cluster of oligonucleotides, a cluster-specific-phasing coefficient corresponding to a nucleotide base for a previous cycle and a cluster-specific-pre-phasing coefficient corresponding to a nucleotide base for a subsequent cycle;
adjust the signal based on the cluster-specific-phasing coefficient and the cluster-specific-pre-phasing coefficient; and
determine a nucleotide-base call for the read position corresponding to the cluster of oligonucleotides based on the adjusted signal.

13. The system of claim 12, further comprising instructions that, when executed by the at least one processor, cause the system to determine, on a sequencing machine of the system, the cluster-specific-phasing coefficient and the cluster-specific-pre-phasing coefficient utilizing a Linear Equalizer, Decision Feedback Equalizer, Maximum Likelihood Sequence Estimator, forward-backward model, or machine learning model.

14. The system of claim 12, further comprising instructions that, when executed by the at least one processor, cause the system to determine the cluster-specific-phasing coefficient and the cluster-specific-pre-phasing coefficient after a sequencing run.

15. The system of claim 12, further comprising instructions that, when executed by the at least one processor, cause the system to:
determine, for a set of clusters of oligonucleotides, one or more of a multi-cluster-phasing coefficient for estimated phasing or a multi-cluster-pre-phasing coefficient for estimated pre-phasing; and
adjust the signal based on one or more of the multi-cluster-phasing coefficient, the cluster-specific-phasing coefficient, the multi-cluster-pre-phasing coefficient, or the cluster-specific-pre-phasing coefficient.

16. The system of claim 12, further comprising instructions that, when executed by the at least one processor, cause the system to adjust the signal by:
determining, for the cluster of oligonucleotides, an additional cluster-specific-phasing coefficient corresponding to an additional nucleotide base for an additional previous cycle;
determining, for the cluster of oligonucleotides, an additional cluster-specific-pre-phasing coefficient corresponding to an additional nucleotide base for an additional subsequent cycle; and
determining a cluster-specific-phasing correction based on the cluster-specific-phasing coefficient, the additional cluster-specific-phasing coefficient, the cluster-specific-pre-phasing coefficient, and the additional cluster-specific-pre-phasing coefficient.

17. The system of claim 12, further comprising instructions that, when executed by the at least one processor, cause the system to adjust the signal based on the cluster-specific-phasing coefficient and the cluster-specific-pre-phasing coefficient by:
generating a previous-cycle weight estimating a phasing effect of the nucleotide base for the previous cycle based on the cluster-specific-phasing coefficient;
generating a subsequent-cycle weight estimating a pre-phasing effect of the nucleotide base for the subsequent cycle based on the cluster-specific-pre-phasing coefficient;
generating a current-cycle weight estimating the phasing effect and the pre-phasing effect for the cycle based on the cluster-specific-phasing coefficient and the cluster-specific-pre-phasing coefficient;
determining a cluster-specific-phasing correction based on the previous-cycle weight, the subsequent-cycle weight, and the current-cycle weight; and
applying the cluster-specific-phasing correction to the signal.

18. A computer-implemented method comprising:
identifying, for a cluster of oligonucleotides, a read position following an error-inducing sequence within one or more nucleotide-fragment reads;
detecting a signal from labeled nucleotide bases within the cluster of oligonucleotides during a cycle corresponding to the read position;
determining, for the cluster of oligonucleotides, a cluster-specific-phasing correction to correct the signal for phasing and pre-phasing;
adjusting the signal based on the cluster-specific-phasing correction; and
determining a nucleotide-base call for the read position corresponding to the cluster of oligonucleotides based on the adjusted signal.

19. The computer-implemented method of claim 18, wherein the error-inducing sequence comprises a sequence of one or more repeated nucleotide bases or a direction-specific sequence motif.

20. The computer-implemented method of claim 18, wherein determining the cluster-specific-phasing correction comprises:
determining, for the cluster of oligonucleotides, a cluster-specific-phasing coefficient corresponding to a nucleotide base for a previous cycle immediately preceding the cycle and a cluster-specific-pre-phasing coefficient corresponding to a nucleotide base for a subsequent cycle immediately following the cycle; and
determining the cluster-specific-phasing correction based on the cluster-specific-phasing coefficient and the cluster-specific-pre-phasing coefficient.
